(19) United States
(12) Patent Application Publication
(10) Pub. No.: US 2024/0358732 A1
(43) Pub. Date: Oct. 31, 2024

(54) Use of Rutin and Rapamycin in the Preparation of Synergistic Chemotherapy Drugs for Tumor Inhibition

(71) Applicant: Binzhou Medical University, Yantai City (CN)

(72) Inventors: Yu Sun, shanghai (CN); Hanxin Liu, Yantai City (CN)

(21) Appl. No.: 18/645,683

(22) Filed: Apr. 25, 2024

(30) Foreign Application Priority Data
Apr. 26, 2023 (CN) 202310493874.4

(51) Int. Cl. A61K 31/7048; A61K 31/136; A61K 31/436; A61P 35/00; G01N 33/50

(52) U.S. Cl. CPC A61K 31/7048; A61K 31/136; A61K 31/436; A61P 35/00; G01N 33/5011

(57) ABSTRACT

The present disclosure provides a use of rutin and rapamycin for preparing a drug combined with a chemotherapeutic drug for tumor inhibition. The present disclosure reveals that rutin can specifically target and inhibit the senescence-associated secretory phenotype (SASP), and it can act synergistically with rapamycin. Rutin and rapamycin show significant synergistic effects of promoting tumor inhibition after being combined with chemotherapeutic drugs, wherein the promoting effect is surprising.

The present disclosure claims the benefit of priority to Patent Application CN 202310493874.4, named “Use of rutin and rapamycin for preparing a drug combined with a chemotherapeutic drug for tumor inhibition” and filed Apr. 26, 2023.

### TECHNICAL FIELD

The present disclosure belongs to the field of biomedicine, and more specifically, the present disclosure refers to use of rutin and rapamycin for preparing a drug combined with a chemotherapeutic drug for tumor inhibition.

### TECHNICAL BACKGROUND

Cell senescence refers to a usually stable and essentially irreversible state of cell cycle arrest in eukaryotic cells, in which proliferating cells become resistant to growth-promoting stimuli, mostly induced by stress signals such as DNA damage. The expression levels of inflammatory cytokines are significantly elevated in senescent cells, a phenomenon known as the senescence-associated secretory phenotype (SASP). Senescent cells can promote the proliferation of adjacent precancerous cells or increase the malignancy of cancer cells by secreting extracellular matrix proteins, inflammation-related factors and cancer cell growth factors, and collectively referred to as SASP factors.

Senescent cells participate in various physiological and pathological processes in vivo through three main pathways: (1) Senescent cells affect the function of the corresponding tissues by the accumulation of gene expression and morphological changes in senescent cells; (2) Senescent cells limit the regenerative potential of stem cells and undifferentiated progenitor cells, leading to a decline in cellular regeneration capacity; (3) Senescent cells not only exhibit growth cycle arrest but also release a large number of cytokines, chemokines, growth factors, and proteases through autocrine and paracrine pathways, affecting the microenvironment of neighboring cells and tissues, leading to and accelerating diseases.

Stimuli such as DNA damage, telomere dysfunction, oncogene activation, and oxidative stress can all induce SASP in cells. The mechanisms involved are closely related to transcriptional cascades, autocrine loops and persistent DNA damage responses. However, overexpression or inhibition of the classical senescent pathways p53 and p16^{INK4A}/Rb does not affect SASP expression. This indicates that, although cell cycle arrest and SASP in senescent cells often occur together, their regulatory pathways do not completely overlap. It has been reported that DNA damage response increases the secretion of SASP factors IL-6 and IL-8 by activating ataxia-telangiectasia mutated, Nijmegen breakage syndrome protein 1 and checkpoint kinase 2. DNA damage response (DDR) is activated immediately after cellular damage, whereas the maturation of SASP in senescent cells takes about a week or even longer.

Additionally, a transient DNA damage response does not induce cellular senescence or SASP, suggesting that mechanisms other than the DNA damage response are involved in the induction of SASP.

Research shows that DDR, p38MAPK and mTOR signaling pathways act as upstream drivers, while NF-κB and c/EBPβ function as downstream transcription factors in the regulation of SASP in senescent cells. The activity of NF-κB and c/EBPβ transcription factors increases during cellular senescence, participating in the regulation of cytokine expression related to stress and inflammatory signals. During cellular senescence, the phosphorylated NF-κB/RelA subunit translocates to the nucleus, binds to the promoters of SASP genes, and regulates the expression of SASP factors. Therefore, NF-κB is often referred to as the major regulator of SASP. Elevated levels of the zinc finger transcription factor GATA4 have been observed in senescent cells of mouse livers, mouse kidneys and aged human brains. GATA4 can influence the expression of SASP-associated genes IL-6, IL-8 and CXCL1 by regulating NF-κB activity in senescent cells. p38MAPK, a member of the serine/threonine protein kinase family, is an important signal transduction molecule. Activation or inhibition of p38MAPK significantly affects SASP formation in senescent cells. p38MAPK is activated a few days after the onset of senescence and indirectly activates NF-κB by activating the mitogen-activated and stress-activated protein kinases MSK1 and MSK2, resulting in the nuclear accumulation of p65 and p50, consistent with the early development of SASP. Senescent cells do not directly secrete the pro-inflammatory factor IL-1α but have high surface levels of IL-1α. This, along with NF-κB, forms a positive feedback loop that promotes the transcription of inflammatory cytokines, establishing and maintaining SASP. mTOR promotes the secretion of SASP factors by regulating IL-1α levels. Although rapamycin does not affect IL-1α mRNA levels, it significantly reduces the expression of IL-1α protein on the surface of senescent cells. mTOR also regulates the downstream signal MAPKAPK2, which affects SASP factor secretion. During cellular senescence, MAPKAPK2 phosphorylates the RNA-binding protein ZFP36L1, limiting its ability to degrade SASP factor transcripts. The transcription factor c/EBPβ is associated with oncogene-induced senescence. During senescence, c/EBPβ is recruited to the IL-6 promoter, directly promoting the transcription of SASP factors. c/EBPβ is also an essential component of the IL-6 positive feedback autocrine loop, activating the inflammatory network of SASP. c/EBPβ is a key regulator in the early spread of SASP. HMGB2 targets c/EBPβ to regulate SASP by inhibiting the spread of heterochromatin and promoting the expression of SASP genes. During cellular senescence, a large amount of HMGB2 binds to chromatin, eliminating the silencing effect of senescence-associated heterochromatin foci (SAHF) on SASP genes, leading to increased expression of IL-8, IL-6, and other factors.

Epigenetic changes influence senescence by affecting DNA damage repair, telomere length, metabolic pathways, or by activating the expression of senescence-associated genes and miRNAs. Various evidence suggests that changes in chromatin state are closely related to the control of cellular senescence. Cells can sense different senescent stimuli, activating signaling pathways that drive changes in chromatin state. However, these changes induced by senescent signals are still largely unknown. Therefore, unraveling the regulatory mechanisms of cellular senescence and its specific phenotypes from a epigenetic perspective, and subsequently revealing key target molecules and signaling pathways, represent a emerging direction in the fields of senescent biology and geriatric medicine. In-depth exploration is urgently needed to provide important scientific foundations and potential interventions for clinical medicine.

Despite increasing experimental support that targeting cellular senescence can simultaneously treat multiple aging-related diseases, such as tumors, rigorous human clinical trials are needed to better assess the benefits and risks of anti-senescence drugs. Although various SASP inhibitors known internationally can significantly weaken SASP, they cannot kill senescent cells in essence. To pharmacologically alleviate the burden of senescent cells, scientists are developing senolytics, which are small molecules, peptides and antibodies designed to selectively clear senescent cells.

In addition to “senolytics” drugs, there is also interest in “senomorphics” drugs in this field. “Senolytics” primarily function by eliminating senescent cells, while “senomorphics” work by modulating the biological properties of senescent cells rather than eliminating them.

In summary, there is still a need to discover more drugs that affect SASP, aiming to provide additional pathways for the prevention and treatment of cellular senescence and tumors.

### SUMMARY OF THE DISCLOSURE

The aim of the present disclosure is to provide a use of rutin and rapamycin for preparing a drug combined with a chemotherapeutic drug for tumor inhibition.

In the first aspect, the present disclosure provides a use of rutin and rapamycin or derivatives thereof, for preparing a composition combined with a chemotherapeutic drug for specifically-targeted inhibiting senescence-associated secretory phenotype, inhibiting tumors and/or reversing cancer resistance; wherein the chemotherapeutic drug is capable of inducing senescence-associated secretory phenotype after administration, comprising mitoxantrone or bleomycin; the derivatives comprise pharmaceutically acceptable salts, esters, isomers, solvates or prodrugs of rutin and rapamycin.

In one or more embodiments, the tumor inhibition comprises inhibition of the tumor per se and inhibition of tumor migration and invasion.

In one or more embodiments, the senescence-associated secretory phenotype is a senescence-associated secretory phenotype caused by DNA damage.

In one or more embodiments, the DNA damage is a DNA damage induced by a chemotherapeutic drug.

In one or more embodiments, in the composition, the rutin or rapamycin or derivatives thereof is also used for: inhibiting expression of the full spectrum senescence-associated secretory phenotype (SASP); preferably, inhibiting expression of the full spectrum SASP without affecting cellular senescence; interfering with the interaction of ATM with HIF1α and TRAF6, inhibiting acute stress-associated phenotype (ASAP); eliminating malignancy of cancer cells conferred by senescent stromal cells through paracrine pathways; increasing apoptosis rate of cancer cells; and/or inhibiting components of the senescence-associated secretory phenotype, comprising IL8, IL6, ILla, IL1b, CXCL3, MMP3 and GM-CSF.

In one or more embodiments, the tumor comprises: prostate cancer, breast cancer, lung cancer, colorectal cancer, gastric cancer, liver cancer, pancreatic cancer, bladder cancer, skin cancer, kidney cancer, esophageal cancer, bile duct cancer, and brain cancer.

In one or more embodiments, the chemotherapeutic drug is mitoxantrone, in the composition (or use in combination), the weight ratio of mitoxantrone to rutin to rapamycin is 1:20˜80:20˜80; preferably, the weight ratio of mitoxantrone to rutin to rapamycin is 1:30˜70:30˜70; more preferably, the weight ratio of mitoxantrone to rutin to rapamycin is 1:40˜60:40˜60 (such as 1:45:45, 1:50:50, 1:55:55, etc.).

In another aspect, the present disclosure provides a pharmaceutical composition or drug kit for specifically-targeted inhibiting senescence-associated secretory phenotype, inhibiting tumors and/or reversing cancer resistance, comprising: rutin and rapamycin or derivatives thereof, and a chemotherapeutic drug; wherein the chemotherapeutic drug is capable of inducing senescence-associated secretory phenotype after administration, comprising mitoxantrone or bleomycin; the derivatives comprise pharmaceutically acceptable salts, esters, isomers, solvates or prodrugs of rutin and rapamycin.

In one or more embodiments, when mitoxantrone, rutin and rapamycin are used in combination, the weight ratio of mitoxantrone to rutin to rapamycin is 1:20˜80:20˜80; preferably, the weight ratio of mitoxantrone to rutin to rapamycin is 1:30˜70:30˜70; more preferably, the weight ratio of mitoxantrone to rutin to rapamycin is 1:40˜60:40˜60 (such as 1:45:45, 1:50:50, 1:55:55, etc.). In another aspect, the present disclosure provides a method of preparing a pharmaceutical composition or drug kit for inhibiting tumors and/or reversing cancer resistance; comprising: mixing rutin and rapamycin or derivatives thereof and a chemotherapeutic drug; or placing rutin and rapamycin or derivatives thereof and a chemotherapeutic drug in the same drug kit; wherein, the chemotherapeutic drug is capable of inducing senescence-associated secretory phenotype after administration, comprising mitoxantrone or bleomycin; preferably, when mitoxantrone, rutin and rapamycin are mixed or used in combination, the weight ratio of mitoxantrone to rutin to rapamycin is 1:20˜80:20˜80; preferably, the weight ratio of mitoxantrone to rutin to rapamycin is 1:30˜70:30˜70; more preferably, the weight ratio of mitoxantrone to rutin to rapamycin is 1:40˜60:40˜60 (such as 1:45:45, 1:50:50, 1:55:55, etc.); the derivatives comprise pharmaceutically acceptable salts, esters, isomers, solvates or prodrugs of rutin and rapamycin.

In one or more embodiments, rutin and rapamycin or derivatives thereof are mixed with a chemotherapeutic agent and divided into unit dosages according to the course of administration

In another aspect, the present disclosure provides a use of rutin and rapamycin or derivatives thereof, for preparing a composition for specifically-targeted inhibiting senescence-associated secretory phenotype; preferably, the rutin and rapamycin or derivatives thereof interfere with the interaction of ATM with HIF1α and TRAF6, inhibit acute stress-associated phenotype; eliminate malignancy of cancer cells conferred by senescent stromal cells through paracrine pathways; and/or, increase apoptosis rate of cancer cells; the derivatives comprise pharmaceutically acceptable salts, esters, isomers, solvates or prodrugs of rutin and rapamycin.

In another aspect, the present disclosure provides a method of screening a potential substance for promoting rutin and rapamycin to target and inhibit senescence-associated secretory phenotype, inhibit tumors and/or reverse cancer resistance, wherein the method comprises:
(1) providing a system of tumor microenvironment, wherein the system comprises tumor cells (preferably it also comprises stromal cells);
(2) treating the system of (1) with a chemotherapeutic drug, inducing senescence-associated secretory phenotype in the tumor microenvironment; wherein the chemotherapeutic drug is capable of inducing senescence-related secretory phenotype (SASP) after administration, comprising mitoxantrone or bleomycin; administering rutin and rapamycin before, during or after inducing the senescence-related secretory phenotype in the tumor microenvironment; and
(3) adding the candidate substance to the system in (2) and observing its effect on the tumor microenvironment system; if the candidate substance statistically promotes (significantly promotes, such as promotes by more than 10%, 20%, 30%, 50%, or more) rutin and rapamycin to inhibit the senescence-associated secretory phenotype, inhibit tumors and/or reverse cancer resistance, then the candidate substance is a potential substance that can be used in combination with rutin and rapamycin to inhibit tumors.

In one or more embodiments, cell apoptosis or the status of the senescence-associated secretory phenotype (SASP) can be evaluated by observing caspase-3 cleavage activity or the expression of SASP factors. Preferably, the SASP factors comprise but are not limited to: IL6, IL8, ILla, IL1b, CXCL3, MMP3, GM-CSF.

In one or more embodiments, it can be evaluated by observing the interaction of ATM with HIF1α and TRAF6 (to inhibit acute stress-associated phenotype). If the ability of rutin and rapamycin interfering with the interaction of ATM with HIF1α and TRAF6 (to inhibit acute stress-associated phenotype) is promoted (significantly promoted, such as, promoted by more than 10%, 20%, 30%, 50% or more), then the candidate substance is a potential substance that can be used in combination with rutin and rapamycin to inhibit tumors.

In one or more embodiments, it can be evaluated by observing malignancy of cancer cells conferred by senescent stromal cells through paracrine pathways. If the candidate substance produces senescent stromal cells and causes them to confer malignancy of cancer cells through paracrine pathways, then the candidate substance is a potential substance that can be used in combination with rutin and rapamycin to inhibit tumors.

In another aspect, the present disclosure provides a method for screening a potential substance for inhibiting senescence-associated secretory phenotype, wherein the method comprises: (1) providing a system of stromal cells, inducing the senescence-associated secretory phenotype in the system; administering rutin and rapamycin before, during or after inducing the senescence-related secretory phenotype in the system; (2) adding the candidate substance to the system of (1) and observing its effect on the system of stromal cells. If the candidate substance statistically promotes rutin and rapamycin to inhibit the senescence-associated secretory phenotype, then the candidate substance is a potential substance that can be used in combination with rutin and rapamycin to inhibit the senescence-associated secretory phenotype.

In one or more embodiments, a control group is also included, so as to clearly distinguish the difference of the system of tumor microenvironment in testing group and that in the control group, or the difference of rutin to target and inhibit senescence-associated secretory phenotype, inhibit tumors and/or reverse cancer resistance and that in the control group.

In one or more embodiments, the potential substance comprises (but are not limited to): small molecule compounds, mixtures (e.g., plant extracts), biological macromolecules, modulating agents for signaling pathway, etc., designed specifically or present in full spectrum compound/biomolecule library.

Other aspects of the present disclosure will be apparent to those skilled in the art based on the disclosure herein.

### DESCRIPTION OF DRAWINGS

FIG. 1. An experimental flow chart and technical route of the screening of a natural medicinal agent (NMA) library to obtain drugs or materials with anti-senescence potential.

FIG. 2. Various candidates of natural library were screened using human stromal cells PSC27 in vitro for analyzing the effect of specific drugs on the viability of proliferating cells and senescent cells. A blue dot in the pink rectangular region represents a drug of senolytics.

FIG. 3. Assessment of the effects of 18 candidates in NMA library after PSC27 cell treatment on the survival of proliferating cells and senescent cells. CTRL, proliferating cells. SEN, senescent cells. PCC1, procyanidin C1, as a positive control for senolytics.

FIG. 4. Assessment of the effects of 19 candidates in NMA library (each applied at a final concentration of 3 μg/mL, with others at different concentrations as indicated in the figure, same in later figures) after PSC27 cell treatment on the survival of proliferating cells and senescent cells. CTRL, proliferating cells. SEN, senescent cells.

FIG. 5. Appraisal of the effects of 19 candidates in NMA library (each applied at a final concentration of 1 μg/mL) on the expression profile of SASP factor IL8 (interleukin 8), in proliferating cells and senescent cells. All data are normalized results compared to the CTRL group. \hat{ }, P>0.05; *, P<0.05; **, P<0.01; ***, P<0.001.

FIG. 6. Appraisal of the effects of 18 candidates in NMA library (each applied at a final concentration of 1 μg/mL) on the expression profile of SASP factor IL8 in proliferating cells and senescent cells. All data are normalized results compared to the CTRL group. \hat{ }, P>0.05; *, P<0.05; **, P<0.01.

FIG. 7. A molecular formula of the natural flavonoid rutin.

FIG. 8. High resolution mass spectra exhibiting the total ion chromatogram (TIC, top) and base peak chromatogram (BPC, bottom) after performance of HPLC-ESI-QTOF-MS.

FIG. 9. The results of SA-β-Gal staining of proliferating human stromal cells PSC27 (early passages of about p10) 7-10 days after they were in vitro treated with chemotherapeutic drug bleomycin (BLEO) at a concentration of 50 μg/ml. Left, representative images; Right, statistical data. CTRL, control cells; BLEO, cells treated with bleomycin. DMSO, solvent; Rutin, rutin. \hat{ }, P>0.05; ****, P<0.0001.

FIG. 10. The results of BrdU staining of PSC27 cells treated with chemotherapeutic drug bleomycin (BLEO) at a concentration of 50 μg/ml. Left, representative images; Right, statistical data. CTRL, control cells; BLEO, cells treated with bleomycin. DMSO, solvent; Rutin, rutin. \hat{ }, P>0.05; ***, P<0.001.

FIG. 11. The relative expression levels of typical SASP factors in senescent cells induced by BLEO and treated with different concentrations of rutin (20-100 μM) according to the detection and analysis of fluorescent quantitative PCR (qRT-PCR). All data are normalized results compared to the CTRL group. *, P<0.05; **, P<0.01; ***, P<0.001.

FIG. 12. A heatmap depicting the transcriptomic expression landscape of PSC27 cells upon three conditions by bioinformatical analysis. CTRL, control cells; BLEO, cells treated with bleomycin; B/R, senescent cells treated with rutin. Red asterisk, SASP protein factor.

FIG. 13. The results of GSEA analysis showed that the expressions of SASP factors are intensively upregulated in BLEO-induced senescent cells, but significantly decreased after rutin treatment. SASP signature, SASP molecule signature.

FIG. 14. A heatmap depicting the transcriptomic expression landscape of PSC27 cells upon senescence induction and rutin intervention by bioinformatical analysis. Note, a total of 3733 genes were down-regulated and a total of 886 genes were up-regulated, respectively (P<0.01, Fold change>4).

FIG. 15. Representative functions of molecular functions for 100 molecules with significant down-regulation caused by rutin in senescent cells according to the KEGG pathway analysis.

FIG. 16. Representative components of cellular components for 100 molecules with significant down-regulation caused by rutin in senescent cells according to the KEGG pathway analysis.

FIG. 17. Representative processes of biological processes for 100 molecules with significant down-regulation caused by rutin in senescent cells according to the KEGG pathway analysis.

FIG. 18. Immunoblot analysis of DDR pathways and SASP expression of PSC27 cells upon BELO and/or rutin intervention. GAPDH, protein loading control.

FIG. 19. Protein interactions of PSC27 cells upon BELO and/or rutin intervention were analyzed by co-immunoprecipitation (TAK 1 antibody-mediated) and immunoblot assay. Input, total protein lysate. GAPDH, protein loading control.

FIG. 20. Protein interactions of PSC27 cells upon BELO and/or rutin intervention were analyzed by co-immunoprecipitation (ATM antibody-mediated) and immunoblot assay. Input, total protein lysate. GAPDH, protein loading control.

FIG. 21. Immunoblot analysis of cell lysates of PSC27 cells upon BELO and/or rutin intervention after separation of the nucleoplasm. C, cytoplasmic; N; Nuclear. Lamin A/C, loading control for nuclear proteins. GAPDH, loading control for cytoplasmic proteins.

FIG. 22. The relative expression levels of a group of typical SASP molecules in senescent cells induced by BLEO and treated with PX-478 or C25-140 according to the detection and analysis of fluorescent quantitative PCR (qRT-PCR). All data are normalized results compared to the CTRL group. \hat{ }, P>0.05; *, P<0.05; **, P<0.01; ***, P<0.001; ****, P<0.0001.

FIG. 23. Measurement of Reactive Oxygen Species (ROS) production in PSC27 cells under various conditions with DCFH-DA, a fluorescent reagent. Left, representative images, scale bar, 20 m; Right, comparative statistics.

FIG. 24. Proliferation assay of prostate cancer cells cultured in vitro. PC3, DU145, M12 and LNCaP were cultured with conditioned media collected from PSC27 cells under different conditions, respectively. **, P<0.01; ***, P<0.001. Scale bar, 100 m. Rapamycin, rapamycin. RR, Rutin/Rapamycin.

FIG. 25. Migration assay of prostate cancer cells cultured in vitro. PC3, DU145, M12 and LNCaP were cultured with conditioned media collected from PSC27 cells under different conditions, respectively. **, P<0.01; ***, P<0.001; ****, P<0.0001. Scale bar, 100 m. RR, Rutin/Rapamycin.

FIG. 26. Invasion assay of prostate cancer cells cultured in vitro. PC3, DU145, M12 and LNCaP were cultured with conditioned media collected from PSC27 cells under different conditions, respectively. **, P<0.01; ***, P<0.001; ****, P<0.0001. Scale bar, 100 μm. RR, Rutin/Rapamycin.

FIG. 27. Drug resistance of prostate cancer cells cultured in vitro. PC3, DU145, M12 and LNCaP were cultured with conditioned media collected from PSC27 cells under different conditions, respectively. **, P<0.01; ***, P<0.001; ****, P<0.0001. IC50, half inhibitory concentration. RR, Rutin/Rapamycin.

FIG. 28. Dose-response curves (nonlinear regression/curve fit). PC3 cells were cultured with the CM of PSC27 native based on chemotherapeutic drugs or induced senescent by BLEO (PSC27-BLEO), and concurrently treated by a wide range of concentrations rutin.

FIG. 29. PC3 mixed with PSC27 cells of the CTRL group or BLEO injury group were transplanted into the subcutaneous tissue of mice to form transplanted tumors. At the end of the 8th week, the mice were dissected and the tumors were obtained, with the volumes of the tumors under the conditions of each group detected and compared. \hat{ }, P>0.05; ***, P<0.001; ****, P<0.0001.

FIG. 30. A schematic diagram of the administration of mice in the preclinical trial. Human stromal cells PSC27 and cancer cells PC3 were mixed in vitro (1:4) and then transplanted into mice subcutaneously to form transplanted tumors. After multiple treatment cycles under the condition of single drug or combined drug administration, the mice were finally sacrificed, and the expression changes of relevant molecules in tumor tissues were analyzed pathologically.

FIG. 31. A schematic diagram of the administration time and administration method for the mice in the preclinical trial. Every two weeks was a dosing cycle, and MIT (mitoxantrone) was intraperitoneally administered to the mice on the first day of the 3rd/5th/7th weeks. From the first day of the 5th week, the mice were intraperitoneally administered with rutin, once a week. After the 8-week course of treatment, the mice were dissected for pathological identification and expression analysis.

FIG. 32. The tumor terminal volume statistical analysis. Chemotherapeutic drug MIT alone or together with anti-senescence drug rutin was used to administer mice, and after the 8th week, the tumor size of each group was compared and analyzed. Left, statistical analysis. Right, representative images of tumors in each group

FIG. 33. Varying expression levels of transcripts between the groups of a set of typical SASP molecules in tumor-bearing sites of mice according to the detection and analysis of fluorescent quantitative PCR (qRT-PCR). Data in each group is normalized for the sample with the lowest expression value within the group. IL6, IL8, AREG and ILla are included.

FIG. 34. Varying expression levels of transcripts between the groups of another set of typical SASP molecules in tumor-bearing sites of mice according to the detection and analysis of fluorescent quantitative PCR (qRT-PCR). Data in each group is normalized for the sample with the lowest expression value within the group. MMP1, MMP3, ANGPTL4 and SPINK1 are included.

FIG. 35. Varying expression levels of transcripts between the groups of a set of cellular senescence-specific biomarkers in tumor-bearing sites of mice according to the detection and analysis of fluorescent quantitative PCR (qRT-PCR). Data in each group is normalized for the sample with the lowest expression value within the group. p16^{INK4a} and p21^{CIP1} are included.

FIG. 36. Comparison of cell senescence in PC3/PSC27 tumor-bearing animal lesions in preclinical experiments. Representative pictures after SA-β-Gal staining. Left, representative images. Right, statistical analysis. Scale bar, 100 m.

FIG. 37. A tumor terminal volume statistical analysis. Chemotherapeutic drug MIT alone or together with anti-senescence drug rapamycin was used to administer mice, and after the 8th week, the tumor size of each group was compared and analyzed. Left 6 groups, PC3 cell only; Right 6 groups, PC3 cells and PSC27 cells together formed a reconstituted tissue and transplanted. \hat{ }, P>0.05; **, P<0.01; ***, P<0.001; ****, P<0.0001.

FIG. 38. A tumor terminal volume statistical analysis. Chemotherapeutic drug MIT alone or together with anti-senescence drug Vitamin C was used to administer mice, and after the 8th week, the tumor size of each group was compared and analyzed. Left 6 groups, PC3 cell only; Right 6 groups, PC3 cells and PSC27 cells together formed a reconstituted tissue and transplanted. \hat{ }, P>0.05; **, P<0.01; ***, P<0.001; ****, P<0.0001.

FIG. 39. A tumor terminal volume statistical analysis. Chemotherapeutic drug VCR alone or together with anti-senescence drug rutin and Vitamin C was used to administer mice, and after the 8th week, the tumor size of each group was compared and analyzed. Left 6 groups, PC3 cell only; Right 6 groups, PC3 cells and PSC27 cells together formed a reconstituted tissue and transplanted. \hat{ }, P>0.05; **, P<0.01; ***, P<0.001; ****, P<0.0001. VCR, vincristine.

FIG. 40. A ratio of DNA damage and apoptosis (Caspase 3 cleaved) in mice of each group was analyzed after specific separation of cancer cells in the lesion by LCM technology. \hat{ }, P>0.05; *, P<0.05; **, P<0.01; ***, P<0.001. RR, Rutin/Rapamycin.

FIG. 41. Circulating serum proteins of SASP typical factors in mice were examined using ELISA. Left, AREG. Right, EREG. RR, Rutin/Rapamycin.

FIG. 42. A tumor terminal volume statistical analysis of the reconstituted tissue formed by breast cancer cells MDA-MB-231 and breast stromal cells HBF1203. Chemotherapeutic drug MIT alone or together with rutin and anti-senescence drug rapamycin was used to administer mice, and after the 8th week, the tumor size of each group was compared and analyzed. Left 6 groups, MDA-MB-2313 cells only; Right 6 groups, MDA-MB-231 cells and HBF1203 cells together formed a reconstituted tissue and transplanted. \hat{ }, P>0.05; *, P<0.05; **, P<0.01; ***, P<0.001; ****, P<0.0001. DOX, doxorubicin.

FIG. 43. A tumor terminal volume statistical analysis of the reconstituted tissue formed by breast cancer cells MDA-MB-231 and breast stromal cells HBF1203. Chemotherapeutic drug MIT alone or together with rutin and anti-senescence drug rapamycin was used to administer mice, and after the 8th week, the tumor size of each group was compared and analyzed. Left 6 groups, MDA-MB-2313 cells only; Right 6 groups, MDA-MB-231 cells and HBF1203 cells together formed a reconstituted tissue and transplanted. \hat{ }, P>0.05; *, P<0.05; **, P<0.01; ***, P<0.001; ****, P<0.0001. VIN, vinblastine.

FIG. 44. A comparative analysis of mice body weight data at the end of the course of treatment under various administration conditions in preclinical. \hat{ }, P>0.05. RR, Rutin/Rapamycin.

FIG. 45. A comparative analysis of mice serological data at the end of the course of treatment under the above various administration conditions. Creatinine, urea (kidney indicator), ALP and ALT (liver indicator) data were compared in parallel. \hat{ }, P>0.05. RR, Rutin/Rapamycin.

FIG. 46. A comparative analysis of body weight data of immune intact mice (C57BL/6J) at the end of the course of treatment under various administration conditions. \hat{ }, P>0.05. RR, Rutin/Rapamycin.

FIG. 47. A comparative analysis of serological data of immune intact mice (C57BL/6J) at the end of the course of treatment under various administration conditions. Creatinine, urea (kidney indicator), ALP and ALT (liver indicator) data were compared in parallel. \hat{ }, P>0.05. RR, Rutin/Rapamycin.

FIG. 48. A comparative analysis of blood cell counts of immune intact mice (C57BL/6J) at the end of the course of treatment under various administration conditions in preclinical. WBC, lymphocyte and neutrophil were compared in parallel. \hat{ }, P>0.05. RR, Rutin/Rapamycin.

### DETAILED DESCRIPTION

After extensive and intensive studies, the present inventors reveal that rutin can specifically target and inhibit the senescence-associated secretory phenotype (SASP), and it can act synergistically with rapamycin. Rutin and rapamycin show significant synergistic effects of promoting tumor inhibition after being combined with chemotherapeutic drugs, wherein the promoting effect is surprising.

In the present disclosure, the term “individual”, “subject” or “patient” generally refers to a mammal, especially a human.

In the present disclosure, typical “SASP factors” comprise IL6, IL8, AREG, ILla, MMP1, MMP3, ANGPTL4, SPINK1, and so on.

Herein, rutin and rapamycin can also be used to reduce resistance to cancer therapy in a patient. The cancer therapy includes chemotherapy or radiation therapy; examples of chemotherapy include cytotoxic therapy such as MIT therapy and examples of radiation therapy include ionizing radiation, mainly including therapies with α-rays, β-rays, γ-rays and X-rays as well as proton and neutron flows.

In the present disclosure, the term “administering” or “giving” refers to providing a compound or pharmaceutical composition of the present disclosure to a subject suffering from a disease or condition to be treated or prevented or at risk of a disease or condition.

In the present disclosure, the term “comprise” means that various components can be used together in the mixture or composition of the present disclosure. The term “mainly consist of . . . ” and “consist of . . . ” is included in the term “comprise”.

### Rutin/Rapamycin

After preliminary extensive screening studies, the inventors found that rutin can inhibit the expression of full spectrum of SASP and inhibit SASP components IL6, IL8, ILla, IL1b, CXCL3, MMP3 and GM-CSF, demonstrating significant potential for application in inhibiting the gene expression closely related to the pro-inflammatory response and secretory activity of senescent cells. Meanwhile, rutin inhibits the acute stress-related phenotype (ASAP) by interfering with the interaction of ATM with HIF1α and TRAF6. Studies have also shown that rutin can eliminate malignancy of cancer cells conferred by senescent stromal cells through paracrine pathways.

Furthermore, the inventors found that rutin and rapamycin can act synergistically, and when combined with chemotherapeutic drugs, they can effectively exert benign complementary effects for targeting lesions, achieving surprising synergistic effects.

The structural formula of the rutin is as follows:

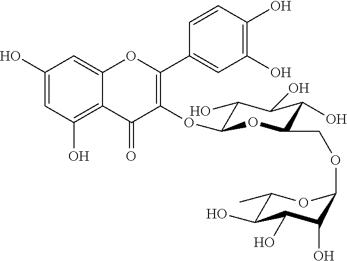

The molecular formula is C51H79NO13 structural formula is as follows:

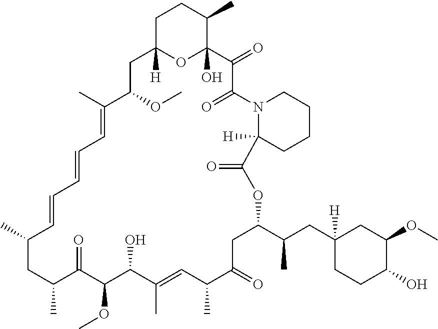

In the present disclosure, “compound” (including rutin, rapamycin, salt or prodrug thereof, etc.) can be a compound in pure form, or a compound with a purity greater than 85% (preferably greater than 90%, such as 95%, 98%, 99%).

Those skilled in the art should understand that, after knowing the structure of the compound of the present disclosure, the compound of the present disclosure can be obtained by various methods well known in the art, by using known raw materials in the art, such as methods of chemical synthesis or extraction from organisms, these methods are all included in the present disclosure. In addition, rutin or rapamycin may also be a commercial product.

In the present disclosure also comprises a pharmaceutically acceptable salt, ester, isomer, solvate or prodrug of the rutin or rapamycin, as long as they retain the same or substantially same functions with the compound of rutin or rapamycin. In the present disclosure, a “pharmaceutically acceptable” component is one that is suitable for use with humans and/or animals without undue adverse side effects (such as toxicity, irritation, and allergic response) commensurate with a reasonable benefit risk ratio. The “pharmaceutically acceptable salt” can be an acid salt or basic salt of the rutin or rapamycin.

“Pharmaceutically acceptable acid salt” refers to a salt that can maintain the biological activity and properties of the free base, and such salt will not have undesired biological activity or other changes. Such salts may be formed from inorganic acids such as, but not limited to, hydrochloric acid, hydrobromic acid, sulfuric acid, nitric acid, phosphoric acid, and the like. Such salt may also be formed from an organic acid, for example, but not limited to, acetic acid, dichloroacetic acid, adipic acid, alginic acid, ascorbic acid, aspartic acid, benzenesulfonic acid, benzoic acid, 4-acetamidobenzoic acid, camphoric acid, camphorsulfonic acid, capric acid, caproic acid, caprylic acid, carbonic acid, cinnamic acid, citric acid, cyclamic acid, dodecylsulfonic acid, and so on.

“Pharmaceutically acceptable basic salt” refers to a salt that can maintain the biological activity and properties of the free acid, and such salts will not have undesired biological activity or other changes. These salts are prepared by adding an inorganic or organic base to the free acid. Salts derived from inorganic bases include, but are not limited to, slats of sodium, potassium, lithium, ammonium, calcium, magnesium, iron, zinc, copper, manganese, aluminum and the like. Preferred inorganic salts are slats of ammonium, sodium, potassium, calcium and magnesium. Salts derived from organic bases include, but are not limited to, primary, secondary, and tertiary ammonium salts. Substituted amines include naturally substituted amines, cyclic amines, and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benzamine, N′-dibenzylethylenediamine, ethylenediamine, glucosamine, methylglucamine, theobromine, triethanolamine, bradykinin, purine, piperazine, piperidine, N-ethylpiperidine, polyamide resin, and the like. Preferred organic bases are isopropylamine, diethylamine, ethanolamine, trimethylamine, dicyclohexylamine, choline and caffeine.

The compound disclosed in the present disclosure may exist as a solvate (such as a hydrate), including monohydrate, dihydrate, hemihydrate, sesquihydrate, trihydrate, tetrahydrate, and similar structures. In the present disclosure, a prodrug of the rutin or rapamycin is also included, and the “prodrug” refers to a compound that undergoes metabolism or chemical reaction in the body of a subject and converts into the desired rutin or rapamycin after being taken in an appropriate way.

In the present disclosure, isomers of rutin or rapamycin are also included. This is because compounds have one or more asymmetric centers, and so these compounds may exist as racemic mixtures, individual enantiomers, individual diastereomers, diastereomeric mixtures, cis- or trans-isomers.

Those skilled in the art should understand that, after knowing the structure of the compound of the present disclosure, the compound of the present disclosure can be obtained by various methods well known in the art, by using known raw materials in the art, such as methods of chemical synthesis or extraction from organisms (e.g., animals or plants), these methods are all included in the present disclosure. The compounds of the present disclosure can be synthesized by methods known in the art. The synthesized compounds can be further purified by methods such as column chromatography, high-performance liquid chromatography, and so on. In addition, compounds of the present disclosure are commercially available.

### Rutin and Rapamycin and Combined Use with a Chemotherapeutic Drug

As previously mentioned, the inventors have discovered that the combined use of rutin and rapamycin with certain specific chemotherapeutic drugs can effectively exhibit a benign complementary effect targeted to the disease, resulting in remarkably significant synergistic effects. Preferably, the chemotherapeutic drug is capable of inducing senescence-associated secretory phenotype after administration, comprising mitoxantrone or bleomycin; the derivatives comprise pharmaceutically acceptable salts, esters, isomers, solvates or prodrugs of rutin or rapamycin.

During screening of drugs that inhibit SASP expression, the present inventors found that a handful of NMA components exhibited a remarkable senomorphic potential, wherein rutin is prominent. Further screening shows that the synergistic effect of rutin and rapamycin is more prominent.

Rapamycin or a derivative thereof is mainly used as an immunosuppressive drug in the prior art. Rapamycin inhibits cell cycle G0 and G1 phases by binding to the corresponding immunophilin RMBP and blocks G1 into S phase to exert the functions, with the following effects: (1) inhibiting T and B cell proliferation; (2) inhibiting IL-1, IL-2, IL-6 and IFN-gamma induced lymphocyte proliferation; (3) inhibiting IgG and donor-specific antibody (cytotoxic antibody) production; (4) inhibiting monocyte proliferation. Previous work of the present inventors also includes studies of rapamycin on tumors. However, it has also been found in the art that it has an adverse effect on tumors. Studies have found that it is pro-cancerous, for example, by activating Akt (through IGF-1R and mTORC2, promoting Akt activation in various cancer cells and exacerbating glioma, etc.); it is also considered that it worsens the conditions of pancreatic cancer, liver cancer; rapamycin is also thought to promote cancer via the MAPK/ERK pathway, PDGFRβ/MAPK pathway, MAPK/Mnk/eIF4E pathway, and so on. Thus, there is significant controversy regarding the effects of rapamycin on tumors.

Rutin can dampen expression of the full spectrum SASP without affecting cellular senescence. Rutin has shown significant inhibitory effects on the expression of typical SASP components, including IL6, IL8, ILla, IL1b, CXCL3, MMP3 and GM-CSF. Analysis of RNA-seq datasets to profile the transcriptome-wide expression pattern of PSC27 indicated that, surprisingly, most of the SASP factors were indeed downregulated by rutin, although the expression status of some genes not directly correlated with cellular senescence or the SASP was also altered. GSEA mapping outputs largely confirmed the influence of rutin on senescent cell expression, suggesting a specifically and significantly inhibited SASP. Therefore, rutin and rapamycin have a salient applicable potential in restraining the expression of genes closely correlated with pro-inflammatory response and secretory activity of senescent cells.

Rutin inhibits the ASAP by interfering with the interactions of ATM with HIF1α and TRAF6. The potential target for rutin is downstream of ATM but upstream of TAK1, p38MAPK and other regulatory factors, functionally involving the acute response induced by cellular senescence. Data from p-ATM-mediated IP and corresponding immunoblot analysis consolidated that ATM and TRAF6 can interact with each other, but rutin significantly weakens such an interaction. More importantly, there is a mutual interaction between ATM and HIF1α, which was also subject to interruption by rutin. By regulating the interaction between ATM and its key targets HIF1α and TRAF6 through natural compound rutin, SASP expression can be inhibited while cellular senescence can be maintained. Although rutin did not change the production of ROS in proliferating cells, it significantly suppressed the capacity of ROS generation by senescent cells, thus typically in line with its free radical scavenging ability.

Rutin is capable of eliminating malignancy of cancer cells conferred by senescent stromal cells through paracrine pathways. The inventors studied the effects of rutin on human prostate cancer (PCa) cells. Upon treatment with the CM from senescent PSC27 cells, the inventors observed substantially elevated proliferation of several PCa cell lines PC3, DU145, M12 and LNCaP, accompanied by enhanced migration and invasion. However, these gain-of-functions (migration and invasion) almost completely disappeared upon rutin treatment of cancer cells. The viability of cancer cells substantially increased upon exposure to senescent stromal cell-derived CM, but counteracted by about 80% upon rutin application. Therefore, rutin is able to remarkably deprive cancer resistance of cancer cells to chemotherapeutic drugs conferred by senescent stromal cells. A combination of rutin and rapamycin caused a more pronounced decrease in malignancy of cancer cells based on the inhibitory effect of rutin used alone.

Combination of chemotherapy with rutin improves anti-tumor treatment efficacy. Though MIT per se caused shrinkage of PC3-only tumors, delivery of senomorphics did not show a remarkable effect; these agents did not confer further benefits even when they were combined with MIT, implying the independence of PC3 tumor growth on the tissue-level SASP, specifically in the absence of stromal cells. Strikingly, upon combination of PC3 cells together with their stromal counterparts, the inventors observed markedly increased tumor volumes (p<0.0001), further validating the tumor-promotive effect of stromal cells in vivo. When animals harboring PC3/PSC27 tumors were exposed to MIT, tumor volumes remarkably decreased (35.5%, p<0.001). After dual treatment with the combination of rapamycin or rutin with MIT, the tumors showed further reduction in size, which was extremely significant. Wherein, coadministration of rapamycin and MIT as dual treatments, tumors displayed further shrinkage by 34.1% (p<0.01); coadministration of rutin and MIT as dual treatments, tumors displayed further shrinkage by 48.9% (p<0.0001). This indicates that the combination of rutin with MIT exhibits relatively superior efficacy compared to the combination of rapamycin with MIT. When rapamycin and rutin were used together with the chemotherapeutic drug MIT to treat tumors, the intervention achieved the best effect, with tumor volume further reduced compared to MIT monotherapy (68.3%, p<0.0001), which was unexpected. No such effect was observed when rutin was combined with other drugs, such as the antioxidant vitamin C. The combination of rutin and rapamycin did not achieve the same effect with all chemotherapeutic drugs; for example, no benefit was observed when vincristine (VIN) was used as the chemotherapeutic drug.

MIT per se caused substantial DNA damage and apoptosis in cancer cells. Rutin alone caused neither a typical DDR nor enhanced cell death in PC3/PSC27 xenografts, suggesting limited responses of these tumors when animals were exposed to rutin only. Upon combination with MIT, rutin further increased the percentage of cell apoptosis. When rutin and rapamycin are used in combination with MIT, DNA damage and apoptosis were further enhanced compared to the combination of rutin and MIT. Under this condition, the clearance of cancer cells in the lesions can achieve optimal results.

When rutin was administered with MIT, caspase 3 self cleavage was enhanced. MIT-mediated chemotherapy resulted in elevated levels of circulating AREG and EREG in animals, a pattern that was basically reversed when rutin was delivered. When rutin is combined with rapamycin, it can further reduce the protein levels of AREG and EREG in the blood of mice, compared to the use of rutin alone with MIT.

Results of multi-type tumor studies also showed that the resistance-minimizing effects of the SASP-targeting strategy are not limited to a specific cancer type, but likely applicable to a wide range of solid malignancies.

Based on the new findings of the present inventors, the present disclosure provides a use of rutin and rapamycin or derivatives thereof, for preparing a composition for specifically-targeted inhibiting senescence-associated secretory phenotype (SASP), inhibiting tumors and/or reversing cancer resistance.

As used in the present disclosure, unless otherwise specified, the “tumor” is a tumor that exhibits senescence-associated secretory phenotype in the tumor microenvironment after treatment with genotoxic drugs, and/or is a tumor that develops drug resistance after using genotoxic drugs. For example, it comprises: prostate cancer, breast cancer, lung cancer, colorectal cancer, gastric cancer, liver cancer, pancreatic cancer, bladder cancer, skin cancer, kidney cancer, esophageal cancer, bile duct cancer and brain cancer.

As used in the present disclosure, unless otherwise specified, the “chemotherapeutic drug” is a chemotherapeutic drug induced senescence-associated secretory phenotype (SASP) after administration. Preferably, it is mitoxantrone or bleomycin

In some embodiments of the present disclosure, the “senescence-associated secretory phenotype” is a senescence-associated secretory phenotype causing by DNA damage; preferably, the DNA damage is a DNA damage caused by a chemotherapeutic drug; more preferably, the chemotherapeutic drug comprises a genotoxic drug.

### Drug Screening

After knowing the close correlation and mechanism of rutin and rapamycin and the tumor microenvironment or SASP, this characteristic can be used for screening drugs with further optimized inhibitory effects. Truly useful drugs can be identified from the substances targeting senescent cells in the tumor microenvironment, inhibiting tumors, reversing drug resistance in tumors, or inhibiting/delaying the senescence-associated secretory phenotype. Alternatively, substances from the compounds can also be identified with synergistic effects when combined with rutin and rapamycin.

Therefore, the present disclosure provides a method for screening a potential substance for promoting chemotherapeutic drugs to inhibit tumors, wherein the method comprises: (1) providing a system of tumor microenvironment, wherein the system comprises cancer cells and stromal cells; (2) treating the system of (1) with a chemotherapeutic drug, inducing a senescence-associated secretory phenotype in the tumor microenvironment; (3) adding the candidate substance to the system in (2) and observing its effect on the tumor microenvironment system; if the candidate substance specifically targets and inhibits senescence-associated secretory phenotype and/or promotes the growth of stromal cells (non-senescent cells) (increase the PD rate of stromal cells), then the candidate substance is a potential substance that can be used for promoting chemotherapeutic drugs to inhibit tumors. In step (2), it also comprises: administering rutin and rapamycin before, during or after inducing the senescence-related secretory phenotype in the tumor microenvironment; in step (3), it also comprises: if the candidate substance statistically promotes rutin and rapamycin to clear senescent cells in the tumor microenvironment and/or promotes the growth of stromal cells, then the candidate substance is a potential substance that can be used in combination with rutin and rapamycin to inhibit tumors.

The present disclosure also provides a method for screening a potential substance for inhibiting senescence-associated secretory phenotype, wherein the method comprises: (1) providing a system of stromal cells, inducing the senescence-associated secretory phenotype in the system; administering rutin and rapamycin before, during or after inducing the senescence-related secretory phenotype in the system; (2) adding the candidate substance to the system of (1) and observing its effect on the system of stromal cells. If the candidate substance statistically promotes rutin and rapamycin to inhibit the senescence-associated secretory phenotype, then the candidate substance is a potential substance that can be used in combination with rutin and rapamycin to inhibit the senescence-associated secretory phenotype.

In a preferred embodiment of the present disclosure, when performing screening, a control group may be established to facilitate the observation of indicator changes in the testing group. The control group can be a system without adding the candidate substance, also with other conditions remaining the same of the testing group.

As a preferred embodiment of the present disclosure, the method also comprises: further cell experiments and/or animal experiments on the obtained potential substances, so as to further select and determine substances really useful for inhibiting tumors, reversing drug resistance in tumors or inhibiting/eliminating senescence-associated secretory phenotype.

On the other hand, the present disclosure also provides potential substances obtained by the screening methods for inhibiting tumors, reversing drug resistance in tumors or inhibiting/eliminating senescence-associated secretory phenotype. These preliminary screening substances can constitute a library for screening, so that people can finally screen for really useful drugs.

### Pharmaceutical Composition

The present disclosure provides a pharmaceutical composition, comprising effective amounts (e.g., 0.00001-50 wt %; preferably 0.0001-20 wt %; more preferably 0.001-10 wt %) of the rutin and rapamycin, a chemotherapeutic drug (e.g., 0.000001-20 wt %; preferably 0.00001-10 wt %; more preferably 0.0001-2 wt %) and a pharmaceutically acceptable carrier. In addition, it should be understood that, in need of facilitating clinical administration or clinical treatment, the rutin, rapamycin and the chemotherapeutic drug are not required for mixing and they may be separately contained in separate containers, kits or drug kits, for combined use as desired. The chemotherapeutic drug is capable of inducing senescence-associated secretory phenotype after administration, preferably comprising mitoxantrone or bleomycin.

As used herein, the term “effective amount” refers to an amount that is functional or active for humans and/or animals and is acceptable for administration to humans and/or animals.

As used herein, the “pharmaceutically acceptable carrier” refers to a carrier for the administration of a therapeutic agent, comprising various excipients and diluents. The term refers to pharmaceutical carriers that are, by themselves, not essential active ingredients and are not unduly toxic after administration. Suitable carriers are well known to those of ordinary skill in the art. Pharmaceutically acceptable carriers in compositions may comprise liquids such as water, saline, buffers. In addition, auxiliary substances such as fillers, lubricants, glidants, wetting or emulsifying agents, pH buffering substances and the like may also be present in these carriers. The carrier may also comprise cell transfection reagents. Pharmaceutical forms suitable for injection include: sterile aqueous solution or dispersion and sterile powder (for extemporaneous preparation of sterile injectable solutions or dispersions). In all cases, these forms must be sterile and must be fluid to easily drain from syringe. It must be stable under the conditions of manufacture and storage and must be able to prevent the contaminating effects of microorganisms (such as bacteria and fungi).

As used herein, the “comprising” or “including” includes the terms “containing”, “mainly consisting of” and “consisting of”. The term “mainly consisting of” means that in the composition, besides the major active ingredients (e.g., rutin and rapamycin, the chemotherapeutic drug), minor ingredients and/or impurities in minor amounts that do not affect the active ingredients may be included. For example, sweeteners to improve taste, antioxidants to prevent oxidation, and other additives commonly used in the art may be included.

It can be understood that, after knowing the use of rutin and rapamycin and mechanism in the tumor microenvironment or SASP, various methods well known in the art can be used for administering rutin and rapamycin and/or the chemotherapeutic drug to mammals or humans. These methods are all within the scope of the present disclosure.

Dosage forms of the composition in the present disclosure can be diverse, the dosage form is acceptable as long as the active ingredients can effectively reach the body of the mammal. For example, it may be selected from: injection, tablet, capsule, powder, granule, syrup, solution, suspension, tincture, oral liquid or aerosol.

The effective amount of the in the present disclosure may vary with the mode of administration, the severity of the disease to be treated, and the like. Selection of preferred effective amount can be determined by those skilled in the art based on various factors (e.g. through clinical trials). Such factors comprise but are not limited to: pharmacokinetic parameters (such as bioavailability, metabolism, half-life, and so on) of the rutin and rapamycin, the severity of the disease to be treated, weight, immune status of patients, the route of administration, and so on.

In the specific examples of the present disclosure, some dosing regimens for animals such as mice are given. It is easy for those skilled in the art to convert the dosage of animals such as mice into dosages suitable for humans. For example, it can be calculated according to the Meeh-Rubner formula: A=k×(W^{2/3})/10,000. In the formula, A is the body surface area, calculated in m²; W is the body weight, calculated in g; K is a constant, which varies with animal species. Generally speaking, mice and rats are 9.1, guinea pigs are 9.8, rabbits are 10.1, cats are 9.9, dogs are 11.2, monkeys are 11.8, human is 10.6. It will be understood that, depending on the drug and the clinical situation, the conversion of the administered dose may vary according to the assessment of an experienced pharmacist.

The pharmaceutical compositions of the present disclosure may also be formulated in unit dosages for convenient, on-schedule administration of the drug.

As used herein, the terms “unit dosage”, “unit dosages” refer to dosage(s) that are required to prepare the compositions of the present disclosure for convenient administration in a single administration, including but not limited to, various solid (e.g., tablet) and liquid agents. The unit dosages comprise the compositions of the present disclosure in amounts suitable for single, single day or unit time administration.

In some preferred embodiments of the present disclosure, the composition is in unit dosages. When the composition is prepared in unit dosages, 1 dose composition of the unit dosage is taken every several days or weeks.

The present disclosure also provides a drug kit comprising the pharmaceutical composition or directly comprising the rutin and rapamycin and/or the chemotherapeutic drug. In addition, the kit may also comprise instructions for using the medicine in the kit.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3^{rd} ed. Science Press) or followed the manufacturer's recommendation.

### Materials and Methods

### 1. Cell Culture

### (1) Cell Line Maintenance

The primary normal human prostate stromal cell line PSC27 (obtained from Fred Hutchinson Cancer Research Center) was cultured in an incubator at 37° C. and 5% CO2, and proliferated and passaged in PSCC complete culture medium (American Gibco, or products from Thermo Fisher with the same name).

### (2) Cell Cryopreservation and Recovery

### a. Cell Cryopreservation

Cells in the logarithmic growth phase were collected with 0.25% trypsin, centrifuged at 1000 rpm for 2 min. The supernatant was discarded, and the cells were resuspended in freshly prepared freezing solution. The cells were sub-packaged into labeled sterile cryovials. Then they were cooled by reducing temperature in gradient manner, and finally transferred to liquid nitrogen for long-term storage.

### b. Cell Recovery

The cells frozen in liquid nitrogen were taken out and immediately placed in a 37° C. water bath to allow them to thaw quickly. 2 mL of cell culture medium was added directly to suspend the cells evenly. After the cells adhered to the wall, fresh culture medium was used for replacement.

### (3) In Vitro Experimental Treatment

To cause cell damage, 50 μg/mL bleomycin (bleomycin, BLEO) was added to the culture medium when PSC27 cells grew to 80% (abbreviated as PSC27-CTRL). After 12 hours of drug treatment, the cells were simply washed 3 times with PBS, left in the culture medium for 7-10 days, and then the subsequent experiments were performed.

### 2. Screening of Natural Product Libraries

Pharmacodynamic analysis of a natural product library (NMA) (Shyuanye Biotechnology) comprising a large variety of components, mostly medicinal plant extracts (including some animal-derived substances) with anti-senescence potential. Each product was diluted to a 96-well plate according to a certain concentration gradient, and the density was 5000 cells per well. The medium uses DMEM, and the working concentration of natural product (or compound) is generally controlled at 1 μM to 1 mM. After 3-7 days of drug treatment, cell proliferation was measured with CCK-8 Cell Counting Kit (based on WST-8 principle, Vazyme), and cell apoptosis activity was determined with Caspase 3/7 Activity Kit (Promega).

The initially identified drug candidates were further screened for 30 days. Drugs entering the second-round candidate range were diluted into a 6-well plate with 20,000 cells per well. Medium and drug candidates were changed every other day. In order to determine the effect of each drug on cell phenotype and viability, etc., the confirmatory analysis was performed according to different concentrations of drugs.

### 3. Western Blotting and Immunofluorescence Detection

Cell lysate-derived proteins were separated using NuPAGE 4-12% Bis-Tris gel and transferred to nitrocellulose membranes (Life Technologies). The blot was blocked with 5% skimmed milk for 1 h at room temperature, incubated overnight at 4° C. with the desired primary antibody at the manufacturer's protocol concentration, and then incubated with horseradish peroxidase-conjugated secondary antibody (Santa Cruz) for 1 h. Blot signal detection was carried out with enhanced chemiluminescence (ECL) detection reagent (Millipore) according to the manufacturer's protocol, and ImageQuant LAS 400 Phospho-Imager (GE Healthcare) was used. As a standard protein marker, PageRuler Plus Prestained Protein Ladder (no. 26619) from Thermo Fisher Scientific was used by the inventors.

For immunofluorescent staining, target cells were pre-seeded on a coverslip for at least 24 h after culture in dishes. After a brief wash, the cells were fixed with 4% paraformaldehyde in PBS for 8 min and blocked with 5% normal goat serum (NGS, Thermo Fisher) for 30 min. Mouse monoclonal antibody, anti-phospho-Histone H2A.X (Ser139) (clone JBW301, Millipore) and mouse monoclonal antibody anti-BrdU (Cat #347580, BD Biosciences), and secondary antibody Alexa Fluor®488 (or 594)-conjugated F(ab′)2 were sequentially added to the slides coated with the fixed cells. Nuclei were counterstained with 2 μg/ml of 4′, 6-diamidino-2-phenylindole (DAPI). The most representative image was selected from the three observation fields for data analysis and result display. FV1000 laser scanning confocal microscope (Olympus) was used to acquire confocal fluorescent images of cells.

### 4. Whole-Transcriptome Sequencing Analysis (RNA-Sequencing)

Whole-transcriptome sequencing was performed on the primary human prostate stromal cell line PSC27 under different treatment conditions. Total RNA samples were obtained from stromal cells. Their integrity was verified by Bioanalyzer 2100 (Agilent), RNA was sequenced by Illumina HiSeq X10, and gene expression levels were quantified by software package rsem (https://deweylab.github.io/rsem/). Briefly, rRNA was depleted from RNA samples with the RiboMinus Eukaryote Kit (Qiagen, Valencia, CA, USA); and according to the manufacturer's instructions, TruSeq Stranded Total RNA Preparation Kits (Illumina, San Diego, CA, USA) was used to construct a strand-specific RNA-seq library before deep sequencing.

Paired-end transcriptomic reads were mapped to a reference genome (GRCh38/hg38), and reference-annotation was performed from Gencode v27 using the Bowtie tool. Duplicate reads were identified using the picard Tools (1.98) script to mark duplicates (https://github.com/broadinstitute/picard), and only non-duplicate reads were retained. Reference splice junctions were provided by reference transcriptome (Ensembl Build 73). FPKM values were calculated with Cufflinks, and Cufflinks maximum likelihood estimation function was used to call differential gene expression. Genes with significant changes in expression were defined by false discovery rate (FDR)-corrected P-values<0.05, and only ensembl genes 73 with status “Known” and biotype “coding” were used for downstream analysis.

Next, Trim Galore (v0.3.0) (http://www.bioinformatics.babraham.ac.uk/projects/trim_galore/) was used to trim the reads, while the quality assessment was carried out by using FastQC (v0.10.0) (http://www.bioinformatics.bbsrc.ac.uk/projects/fastqc/). Subsequently, DAVID bioinformatics platform ( ) and ingenuity Pathways Analysis (IPA) program (http://www.ingenuity.com/index.html) were used. A preliminary analysis of the raw data was performed on a free online platform, Majorbio I-Sanger Cloud Platform (www.i-sanger.com) and the raw data were deposited in the NCBI Gene Expression Omnibus (GEO) database with access code GSE156448.

### 5. Protein-Protein Interaction Network Analysis

Protein-protein interaction (PPI) analysis was performed with STRING3.0. Specific proteins that met the criteria were imported into the online analysis software (http://www.networkanalyst.ca), and further hub and module analysis were performed by selecting a minimal interaction network.

### 6. Gene Set Enrichment Analysis (GSEA)

Based on the data obtained from the preliminary analysis of RNA-seq, after analyzing and comparing each differentially expressed significant gene, the genes were sorted using the “wald statistics” obtained from DESeq2. GSEA was performed on the sorting lists of all planning gene sets available in MSigDB (http://software.broadinstitute.org/gsea/msigdb). DESeq2 independent filtering was based on the average of normalized read counts to select genes with very low expression levels. The SASP and GSEA signature were as described in our previous publication (Zhang et al., 2018a).

### 7. Measurement of Gene Expression by Quantitative PCR (RT-PCR)

### (1) Extraction of Total Cellular RNA

RNA was extracted by Trizol reagent. After the RNA was quantified by a spectrophotometer, a small amount of total RNA was taken for 1% agarose electrophoresis to check the status and quality of the RNA.

### (2) Reverse Transcription Reaction

### (3) Real-Time Quantitative PCR Reaction

After the reaction was completed, the amplification of each gene was checked by software analysis, the corresponding threshold cycle number was derived, and the relative expression of each gene was calculated using the 2-ΔΔCt method. The peak and waveform of the melting curve was analyzed to determine whether the obtained amplification product was a specific single target fragment.

### 8. SA-β-Gal Staining

Senescence-associated β-galactosidase (SA-β-Gal) staining was performed following a previously reported procedure (Debacq-Chainiaux et al., 2009). Briefly, cells in culture dishes were washed with PBS and fixed at room temperature. Cells were fixed in 2% formaldehyde and 0.2% glutaraldehyde for 3 minutes. SA-β-Gal was used for staining with freshly prepared staining solution overnight at 37° C. Images were taken on the next day and the percentage of positive cells per unit area was calculated.

### 9. Clonal Expansion Experiment

Single cell Clonal expansion experiment. Briefly, cells were plated in gelatin-coated 12-well plates at a density of 2000 cells/well. Cell clones were counted after crystal violet staining.

### 10. Drug-Induced Apoptosis in Senescent Cells

PSC27 cells were plated in a 96-well dish, and the cells were induced to senescence under 50 μg/ml of BLEO treatment. Rutin and PCC1 were added at concentrations of 100 μM and 50 μM, respectively. Cell culture medium was supplemented with Incucyte Nuclight fast Red Reagent (Essen Bioscience) and Incucyte C-3/7 Apoptosis Reagent (Essen Bioscience). Representative field of view was selected to take pictures.

### 11. Mouse Xenograft Inoculation and Preclinical Treatment Trials

All experimental mouse experiments were performed strictly following the relevant regulations of the experimental animals. Immuno-deficient mice(NOD-SCID mice, ICR) (body weight about 25 g) aged 6-8 weeks were used for animal experiments related to the present patent. Stromal cells PSC27 and epithelial cells PC3 were mixed at a predetermined ratio of 1:4, and each graft contained 1.25×10⁶ cells for tissue remodeling. The xenograft tumors were implanted into mice by subcutaneous transplantation, and the animals were euthanized 8 weeks after the end of the transplantation surgery. Tumor volume was calculated according to the following formula: V=(π/6)×((1+w)/2)³ (V, volume; 1, length; w, width).

In a preclinical treatment trial, subcutaneously transplanted mice were fed with a standard experimental diet, after 2 weeks chemotherapy drugs mitoxantrone (MIT, dose: 0.2 mg/kg) and/or Rutin (500 μl, dose: 10 mg/kg), Rapamycin (500 μl, dose: 10 mg/kg) via intraperitoneal administration. The time points are as follows: all three drugs were administered to mice on the first day of the third, fifth and seventh weeks. A total of 3 cycles of administration was carried out throughout the course of treatment, and each cycle lasted for 2 weeks. After the course of treatment, mouse tumors were harvested for volume measurement and histological analysis. Each mouse cumulatively receives the drug MIT at 0.6 mg/kg body weight, rutin at 30 mg/kg body weight and rapamycin at 30 mg/kg body weight. In order to cause systemic expression of SASP factors induced by chemotherapy, MIT was administered to mice through intravenous infusion according to the above steps and sequence, but the dose was reduced to 0.1 mg/kg body weight/each time (the cumulative dose of MIT received in the whole course of treatment was 0.3 mg/kg body weight) to reduce drug-related toxicity. The chemotherapy experiment ended at the end of the 8th week, and the mice were dissected immediately after sacrifice, and the xenograft tumors were collected and used for pathological system analysis. For breast cancer tumor-bearing mice, the pre-clinical procedures were the same as above, with the chemotherapeutic drugs doxorubicin (DOX, dose: 1.0 mg/kg) and/or Rutin (Rutin) (500 μl, dose: 10 mg/kg), rapamycin (Rapamycin) (500 μl, dose: 10 mg/kg) being administered intraperitoneally. For alternative chemotherapeutics, vincristine or vinblastine (dose: 1.0 mg/kg) is administered intraperitoneally.

### 12. Biostatistical Methods

In the present patent application, all in vitro experiments involving cell proliferation rate, survival rate and SA-β-Gal staining, and in vivo experiments on mouse xenograft tumors and preclinical drug treatment were repeated more than 3 times, and the data were presented in the form of mean±standard error. The statistical analyzes were established on the basis of raw data and calculated by one-way analysis of variance (ANOVA) or a two-tailed Student's t-test, while the results with P<0.05 were considered to be significantly different.

The correlation between factors was tested by Pearson's correlation coefficients. Cox proportional hazards model was used for survival analysis when mice were obtained in several cohorts and grouped in cages. In the model, sex and age were used in the treatment as fixed effects, while cohort and initial cage assignment were used as random effects. Since during the study some mice were moved from their initial cages to minimize stress from the single cage enclosure, we also per-formed analysis without cage effects. The results of the two analyzes did not differ significantly in directionality or statistical significance, which increased confidence in results. Survival analysis was performed using the statistical software R (version 3.4.1; library “coxme”). In most experiments and outcome assessments, investigators made blind selections for assignments. The inventors used baseline body weights to assign mice to experimental groups (to achieve similar body weights between groups), so randomization was only performed within groups matched for body weight. All replicates in the present disclosure were derived from different samples, and each sample was derived from a different experimental animal.

### Example 1. Drug Screening Reveals Rutin as a Potential Small Molecule Senomorphic Agent

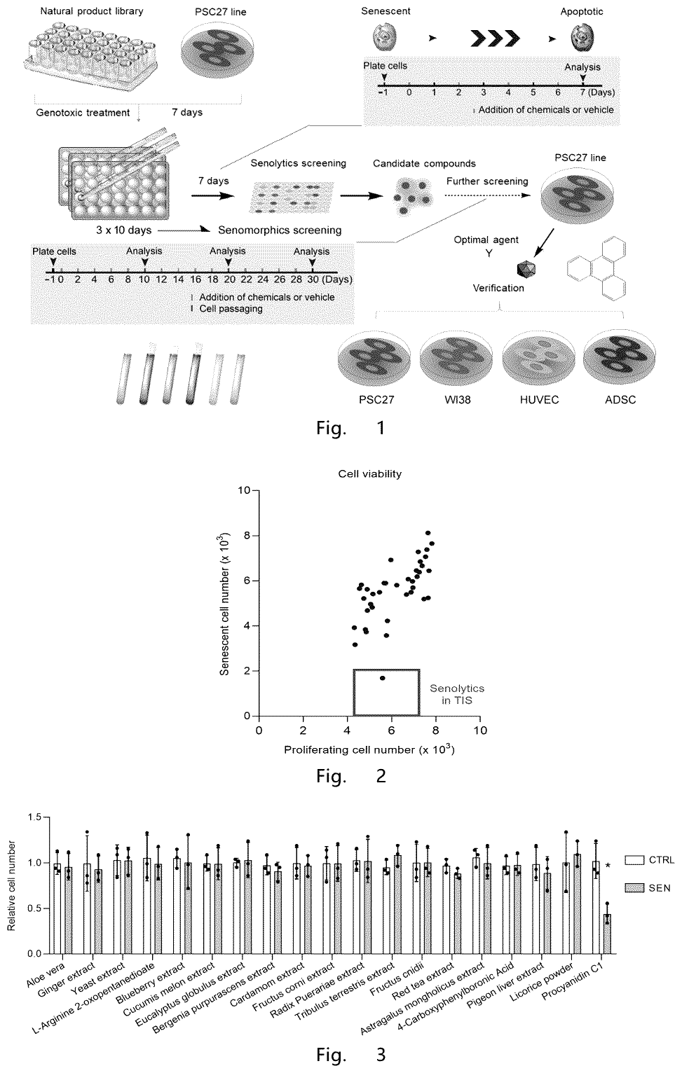

To identify new compounds that can effectively target senescent cells, the inventors performed an unbiased agent screening with a library composed of many natural medicinal agents (NMAs), most of which are phytochemical products. To this end, a primary normal human prostate stromal cell line, PSC27, was selected as a cell-based model by the inventors. Comprising mainly of fibroblasts but with a minor percentage of nonfibroblast cell lineages such as smooth muscle cells and endothelial cells, PSC27 is primary per se and develops a typical SASP upon exposure to stressors such as genotoxic chemotherapy and ionizing radiation. To induce senescence, cells were treated with a pre-optimized sub lethal dose of bleomycin (BLEO) (50 μg/ml) and elevated positivity of senescence-associated β-galactosidase (SA-β-Gal) staining was observed, with reduced BrdU incorporation and augmented DNA damage response (DDR). A screening strategy was set up for comparing the effect of individual medicinal products generated on the survival and expression profile of senescent cells (FIG. 1a).

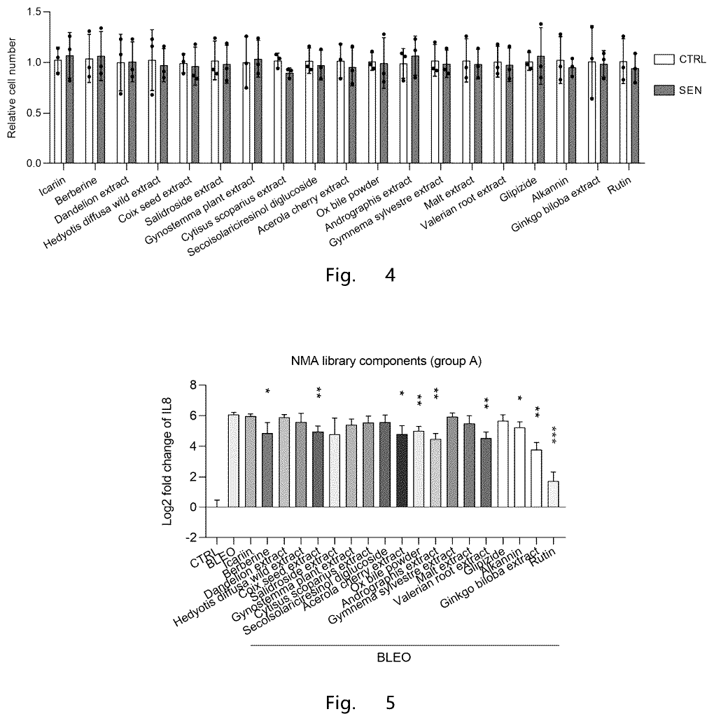

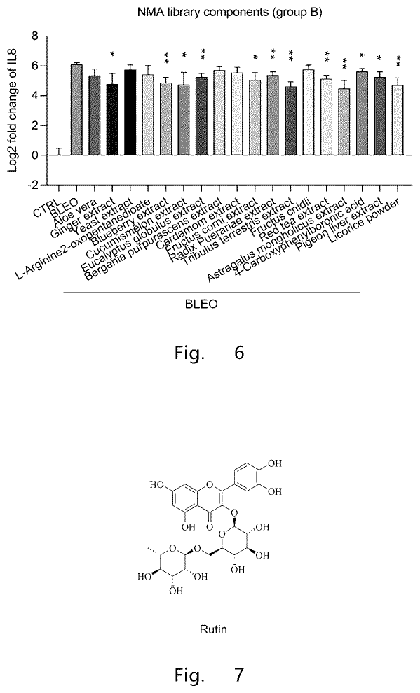

The inventors first determined the efficacy of these NMA components against senescent PSC27, and explored its potential as an experimental cell model for overall drug screening. Preliminary data of the inventors suggested that a number of these compounds were able to alter the bioactivity of senescent (SEN), but not proliferating cells (FIG. 2-4). Such a property implied that PSC27 represents an ideal and qualified model to initiate subsequent procedures, as it allows to selectively target senescent cell populations rather than their growing counterparts, largely securing the feasibility of using this primary stromal line for further investigations. Upon large scale screening of the NMA library, the inventors failed to identify even a single senolytic agent, which was supposed to hold the potential to selectively killsenescent cells in culture like the positive control procyanidin C1(FIG. 2, 3), suggesting the scarcity of such natural senolytics and difficulty in expanding the arsenal of this specific subclass of senotherapeutics.

However, the inventors observed that a handful of NMA components exhibited a remarkable senomorphic potential, which did deserve continued investigations (FIG. 5, 6). And rutin is particularly prominent among them (FIG. 5, 7).

Among the senomorphic agents that showed prominent efficacy in downregulating the expression of interleukin 8 (IL8), a hallmark SASP factor, the inventors selected rutin for analysis in depth (FIG. 7, 8). As a flavonoid derived from wildlife plants, rutin has been reported with a wide range of biological activities. Effects of rutin were considered responsible for the protective effect against several pathological conditions such as hyperglycemia, nephropathy, neuropathy, and cardiovascular disorders. However, its medical implications including therapeutic capacity and functional mechanisms, in modulating the activities of senescent cells, particularly the SASP expression, remain generally unknown and essentially blank.

### Example 2. Rutin Dampens Expression of the Full Spectrum SASP without Affecting Cellular Senescence

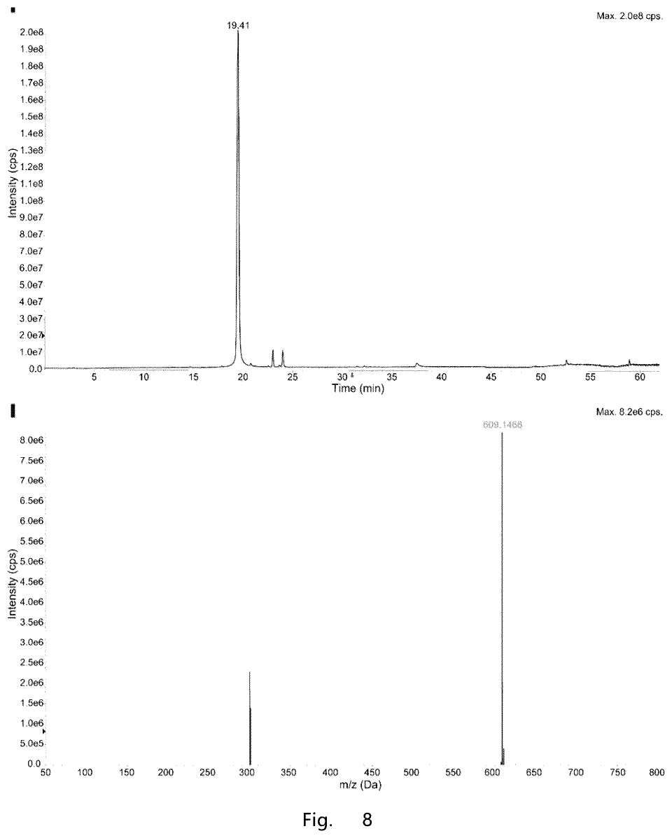

Although rutin inhibits expression of the SASP hallmark factor IL8, whether it affects cellular senescence and a wide spectrum of the SASP, or alternatively, the vast majority of other SASP factors, remains yet unclear. To address these questions, the inventors performed in vitro assays and found that SA-β-Gal staining and BrdU incorporation remained largely unchanged, regardless of proliferating or senescent cells, the latter induced by bleomycin (BLEO), a genotoxic agent frequently administered to cancer patients in clinical oncology (FIG. 9, 10).

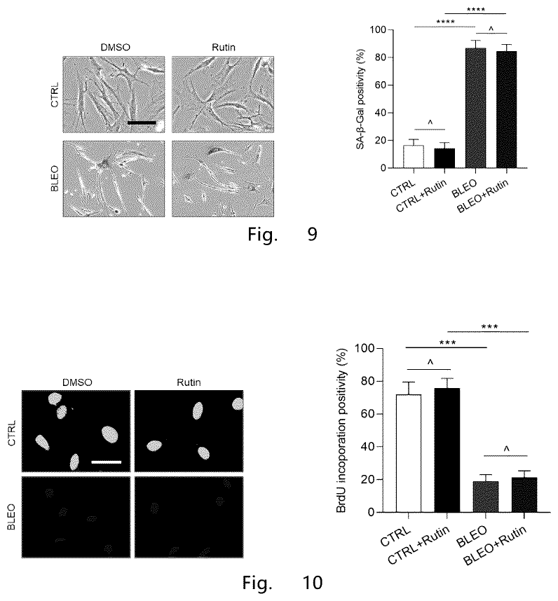

Among the different concentrations the inventors used in culture, 100 μM of rutin seemed to have generated the most optimal effects in restraining the expression of a subset of typical SASP components, including IL6, IL8, ILla, IL1β, CXCL3, MMP3 and GM-CSF (FIG. 11).

Moreover, analysis of RNA-seq datasets to profile the transcriptome-wide expression pattern of PSC27 indicated that most of the SASP factors were indeed downregulated by rutin, although the expression status of some genes not directly correlated with cellular senescence or the SASP was also altered (FIG. 12).

As supporting evidence, GSEA mapping outputs largely confirmed the influence of rutin on senescent cell expression, suggesting a specifically and significantly inhibited SASP (FIG. 13).

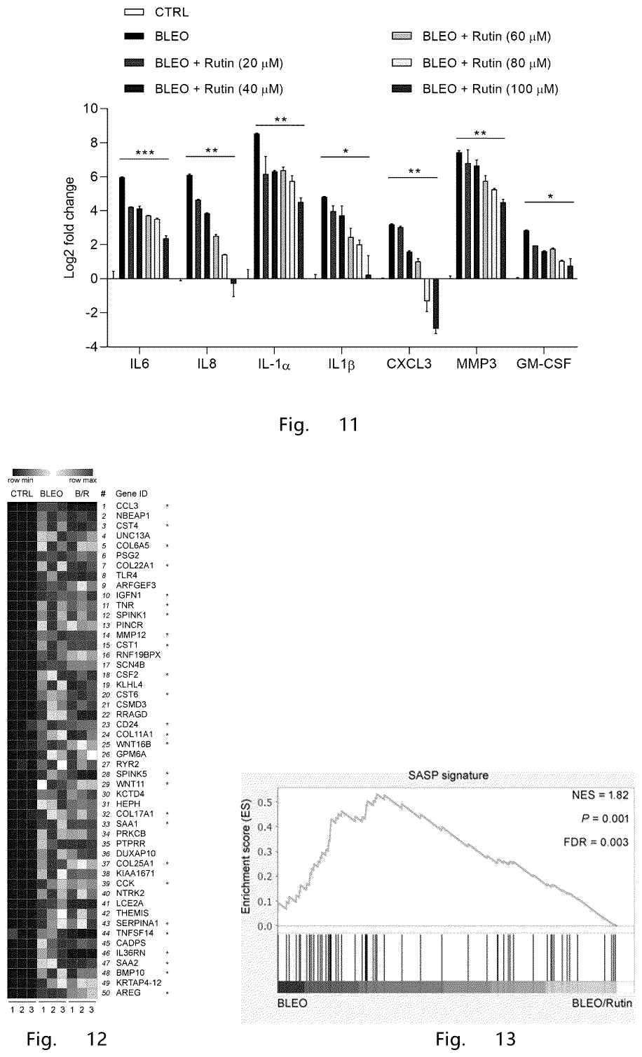

Further analysis by bioinformatics revealed that 4619 transcripts were significantly modified (2-fold change of log 2-based, p<0.01) in senescent PSC27 cells upon exposure to rutin in the culture condition (3733 downregulated and 886 upregulated) (FIG. 14).

After mapping the transcripts to a gene ontology (GO) database comprising HPRD, Entrez Gene, and UniProt accession identifiers, the inventors noticed that the most prevalent molecular functions of upregulated genes (top 100 selected as representatives) were cytokine activity, metallopeptidase activity, structural molecule activity, and receptor activity (FIG. 15).

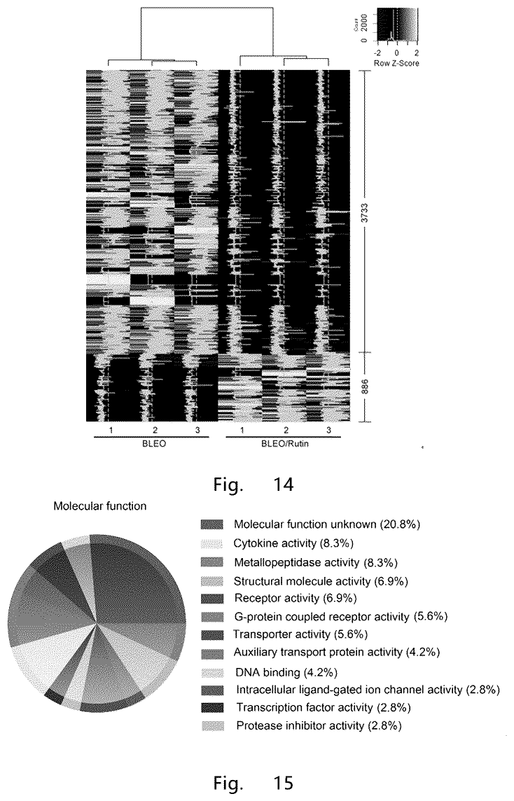

The most typical cellular components of bioactive proteins encoded by these genes were those transported to extracellular niche, followed by those residing in the cytoplasm and nucleus, although many products indeed belong to the subcategories of soluble fraction, extracellular space, extracellular region, and exosomes (FIG. 16). In addition, the most predominant biological processes correlated with the upregulated genes were intercellular communication, inflammatory response, cell growth and/or maintenance, regulation of nucleobase, nucleoside, nucleotide and nucleic acid metabolism (FIG. 17).

Together, data of the inventors suggest a salient capacity of rutin in restraining the expression of genes closely correlated with pro-inflammatory response and secretory activity of senescent cells.

### Example 3. Rutin Inhibits the ASAP by Interfering with the Interactions of ATM with HIF1α and TRAF62.3

Next, the inventors questioned the molecular mechanism supporting rutin to generate influence on the development of cellular senescence-associated phenotypes, particularly the SASP. To assess the potential actions of rutin on intracellular DDR signaling, an event that allows genomic DNA damage to activate downstream inflammatory responses,
the inventors first collected total lysates of PSC27 cells before and after rutin treatment of proliferating or senescent cells. Immunoblot indicated that ATM (one of the central regulators of DDR signaling) was markedly activated upon induction of senescence by BLEO (FIG. 18).

In response to inherent or environmental stimuli, proliferating cells tend to firstly exhibit an acute stress-associated phenotype (ASAP), involving prompt ATM activation and nucleus-to-cytoplasm translocation, TRAF6-mediated mono-ubiquitylation and TAK1 phosphorylation, a process that can be observed within 2-3 days after exposure of cells to insulting damage. As an essential component of genotoxicity stress-induced cellular responses and a key modulator of the ASAP, the cytoplasmic kinase TAK1 was clearly phosphorylated in assays, a change functionally priming it for subsequent engagement in dual feedforward mechanisms to orchestrate the SASP development.

The inventors further noticed that activation of p38MAPK, a downstream target of TAK1, activation of the PI3K/Akt/mTOR axis (mediator of the persistent SASP signaling, indicated by mTOR and AKT phosphorylation), as well as upregulation of IL8, the hallmark factor for ASAP (also for SASP, in most cell lines), took place in an acute manner after BLEO treatment (FIG. 18).

However, in the presence of rutin, these remarkable changes generally diminished, except that ATM phosphorylation remained largely unaffected, suggesting that the potential target of rutin is likely downstream of ATM, but upstream of TAK1, p38MAPK and other regulators, and such a target is functionally implicated in the acute response upon induction of cellular senescence (FIG. 18).

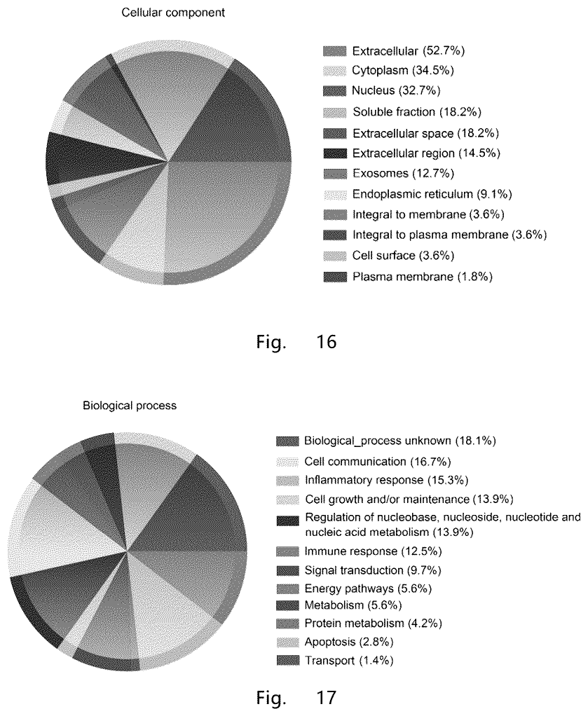

In the course of ASAP, there is a physical association between activated TRAF6 and TAK1, a phenomenon that shows up shortly after DNA damage but subject to suppression by the TAK1 inhibitor, 5Z-7-oxozeaenol. However, whether rutin disrupts this process remains unknown. The inventors thus chose to perform phosphorylated TAK1 (p-TAK1)-mediated IP and subsequent immunoblot analysis, and found that both TAK1 activation and TRAF6-TAK1 interaction were considerably restrained by rutin (FIG. 19). The data confirmed that the direct target(s) rutin should be beyond the physical association of TRAF6 and TAK1.

To establish the mechanism responsible for implications of rutin in the ASAP process, the early response that eventually culminates in the SASP formation as a chronic phenomenon, the inventors performed genome-wide mapping of molecules that hold the potential to interact with ATM. Bioinformatics mining revealed 279 unique ATM interactors and 379 unique TRAF6 interactors in human cells, with 22 interacting molecules shared by both ATM and TRAF6 in the final outputs. Further analysis excluded the necessity of exploring the vast majority of molecules that can interact with both ATM and TRAF6, with hypoxia-inducible factor1α (HIF1a) standing out as a candidate that deserves continued investigation.

Data from p-ATM-mediated IP and corresponding immunoblot analysis consolidated that ATM and TRAF6 can interact with each other, but rutin significantly weakens such an interaction. More importantly, there is a mutual interaction between ATM and HIF1α, which was also subject to interruption by rutin (FIG. 20).

As further evidence, the inventors observed remarkable cytoplasm-to-nucleus translocation of p65 and p50, two master subunits of NF-κB transcriptional complex upon cellular senescence, although this tendency was largely abolished in the presence of rutin (FIG. 21). Of note, HIF1α exhibited nuclear translocation in a manner generally resembling that of p65 and p50 (FIG. 21), suggesting a potential engagement of this factor in modulating the genome-wide expression of genes correlated with senescence and/or senescence-associated phenotypes, specifically the SASP. Like the transcriptional comple NF-B, HIF1α is a key transcription factor for adaptive responses, orchestrating the transcription of numerous genes involved in angiogenesis, erythropoiesis, glycolytic metabolism, and inflammation, while its implication in senescence remains basically underexplored. HIF1α acts as a central factor that transmits ATM-relayed damage signals to nucleus, and may be responsible for upregulation of a series of factors essential for senescence maintenance and the SASP development.

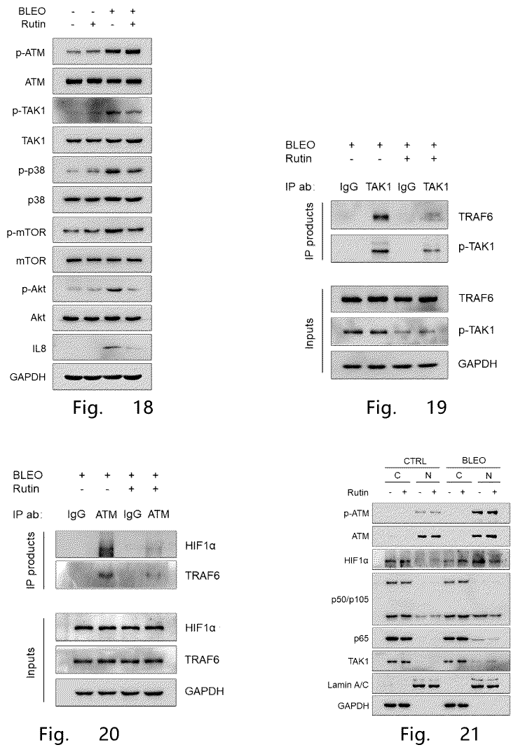

To substantiate this speculation, the inventors assessed the expression of a subset of genes encoding the SASP factors or senescence-specific markers. The data suggested that treatment with either PX-478, a selective HIF-1α inhibitor, or C25-140, a small molecule compound that reduces TRAF6-mediated ubiquitin chain formation, was able to significantly decrease the expression of typical SASP factors examined in the assay (FIG. 22). However, expression of p16^{INK4A} and p21^{CIP1} seemed to remain unaffected. Modulating the interactions between ATM and its key targets specifically HIF1α and TRAF6, as exemplified by the natural agent rutin, holds the potential to suppress the SASP expression, while sustaining cellular senescence, a distinct feature that is largely consistent with the criteria of senomorphics.

In response to genotoxicity delivered by BLEO, PSC27 cells exhibited remarkably elevated ROS levels, in contrast to their normal counterparts (FIG. 23). Although rutin did not change the production of ROS in proliferating cells, it significantly suppressed the capacity of ROS generation by senescent cells, thus typically in line with its free radical scavenging ability.

### Example 4. Rutin Deprives Cancer Cells of Malignancy Conferred by Senescent Stromal Cells in a Paracrine Manner

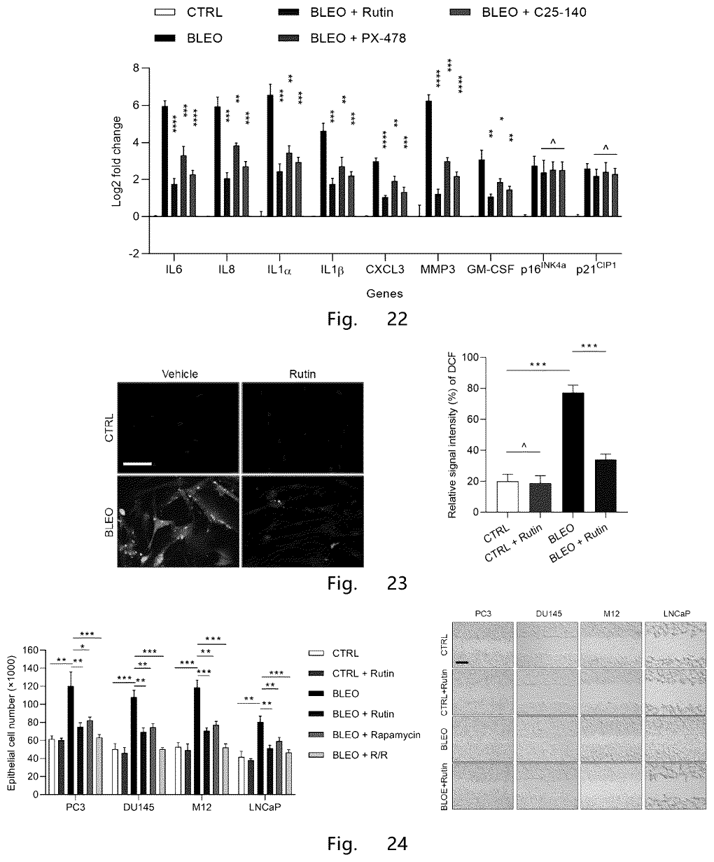

Upon treatment with the CM from senescent PSC27 cells, the inventors observed substantially elevated proliferation of several PCa cell lines PC3, DU145, M12 and LNCaP (p<0.01) (FIG. 24), accompanied by enhanced activities of migration and invasion (FIG. 25, FIG. 26). However, these gain-of-functions almost completely disappeared upon rutin treatment of cancer cells (FIG. 24-FIG. 26). In this assay, the final concentration of rutin is 100 μM. To further enhance the inhibitory effects, the inventors conducted additional drug screening. After analyzing a large number of candidates, rapamycin was ultimately focused because of a significant synergistic effect when combined with rutin. A combination of rutin and rapamycin caused a more pronounced decrease in malignancy of cancer cells based on the inhibitory effects of rutin used alone (FIG. 24-26).

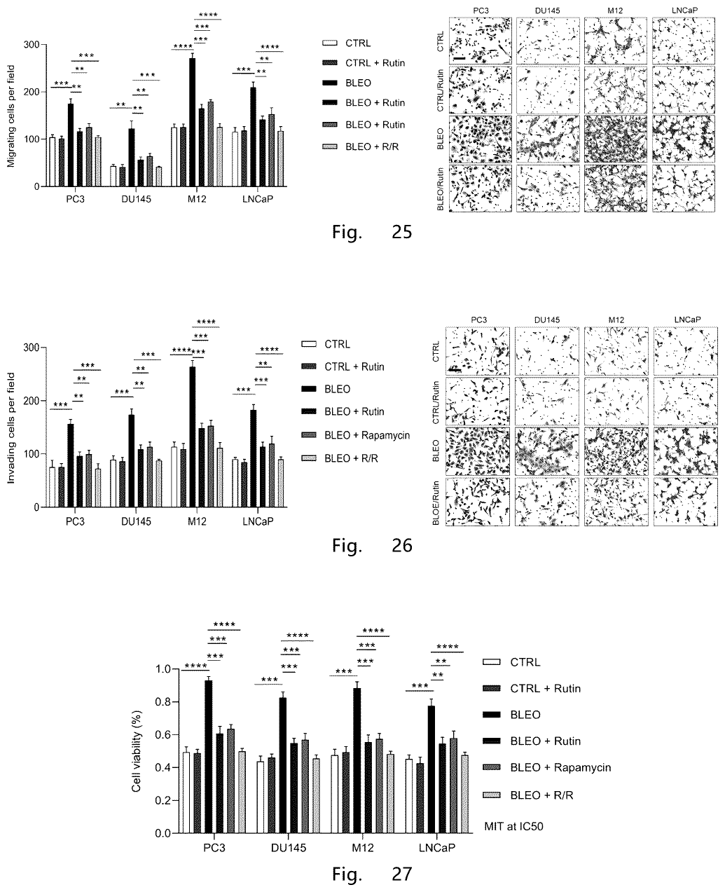

A number of SASP factors including WNT16B, SFRP2, SPINK1 and AREG display strong capacity in conferring resistance to cancer cells. However, whether such a resistance-promoting capacity of the SASP can be prevented by rutin, remains undetermined yet. The inventors found the viability of cancer cells substantially increased upon exposure to senescent stromal cell-derived CM, but counteracted by about 80% upon rutin application (at a final concentration of 100 μM) (FIG. 27).

Although PSC27-BLEO CM increased the viability of PC3 exposed to MIT at 0.1-1.0 μM, a range of doses approaching its serum concentrations in patients under clinical conditions, rutin (at a final concentration of 100 μM) was able to remarkably downregulate cancer resistance conferred by the CM of senescent stromal cells (FIG. 28). Furthermore, surprisingly, the combination of rapamycin and rutin showed better results, essentially reversing the acquired drug resistance of cancer cells to the state of the control group (i.e., the primary PSC27 cells) (according to FIG. 28, the curve almost overlaps with that of the PSC group), which is quite unexpected.

Therefore, rutin was able to remarkably deprive cancer resistance conferred by senescent stromal cells, while this deprivation enhanced when rapamycin and rutin are used in combination because of the synergistic effect. This provides a foundation for developing new therapeutic regimens to improve anticancer efficacy.

### Example 5. Combination of Chemotherapy with Rutin Improves Anti-Tumor Treatment Efficacy in Preclinical Trials

Given the effects of rutin on the expression profile and in vitro phenotypes of cancer cells, the inventors next queried the therapeutic effectiveness that rutin may exhibit under in vivo conditions. To address this problem, the inventors generated tissue recombinants by admixing PSC27 sublines with PC3 cells at a preoptimized ratio (1:4) before subcutaneous implantation to the hind flank of experimental mice with severe combined immunodeficiency (SCID). Animals were measured for tumor size at the end of an 8-week period. Compared with tumors comprising PC3 and PSC27 primary naive cells (PSC27^{Naive}), xenografts composed of PC3 and PSC27^{SEN} exhibited significantly enlarged sizes (FIG. 29). However, pretreatment of PSC27^{SEN} cells in vitro prior to generation of tissue recombinants resulted in considerably reduced tumor volumes (p<0.0001), largely reproducing the effectiveness of rapamycin in treating tumors, which can generate a long term in vivo consequence even after used to treat senescent human cells for once in vitro.

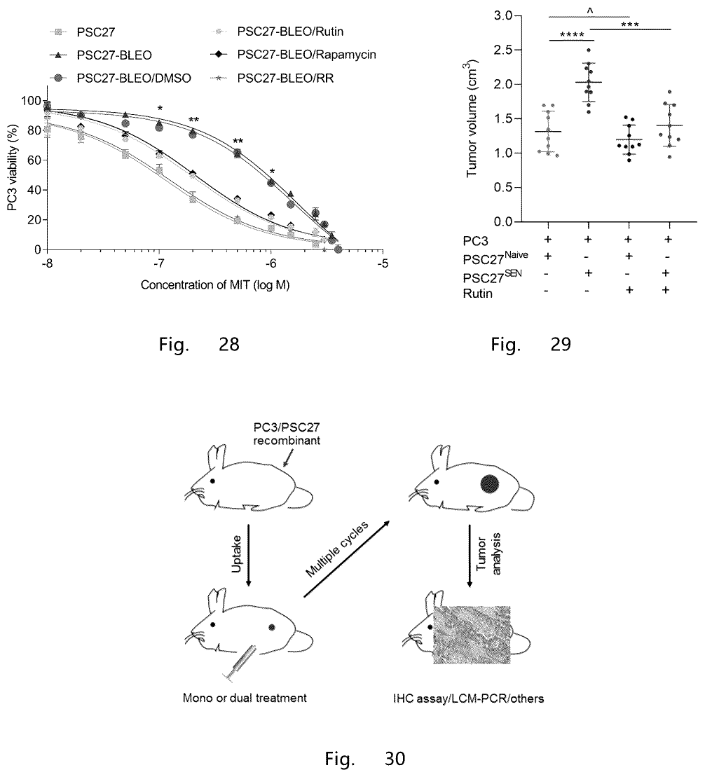

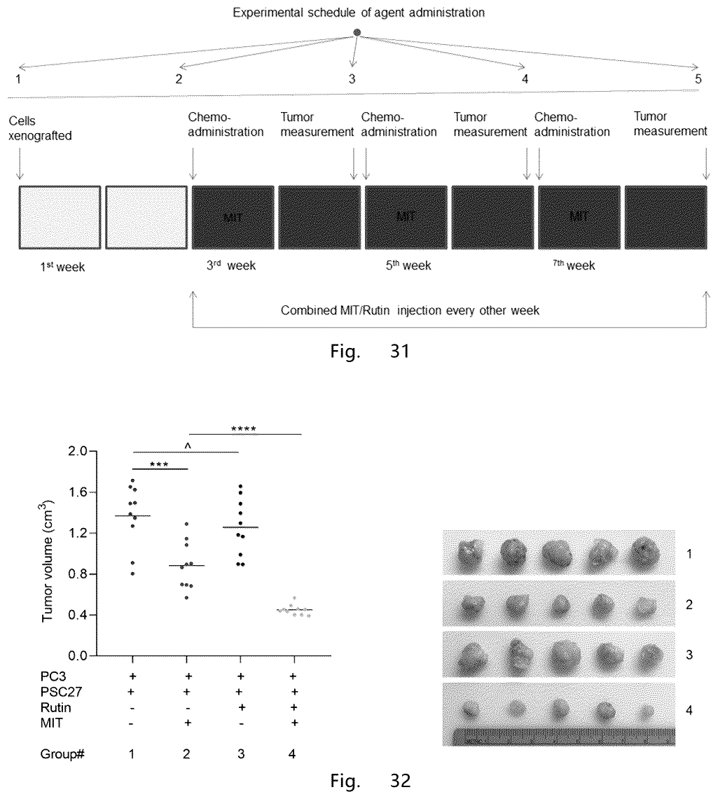

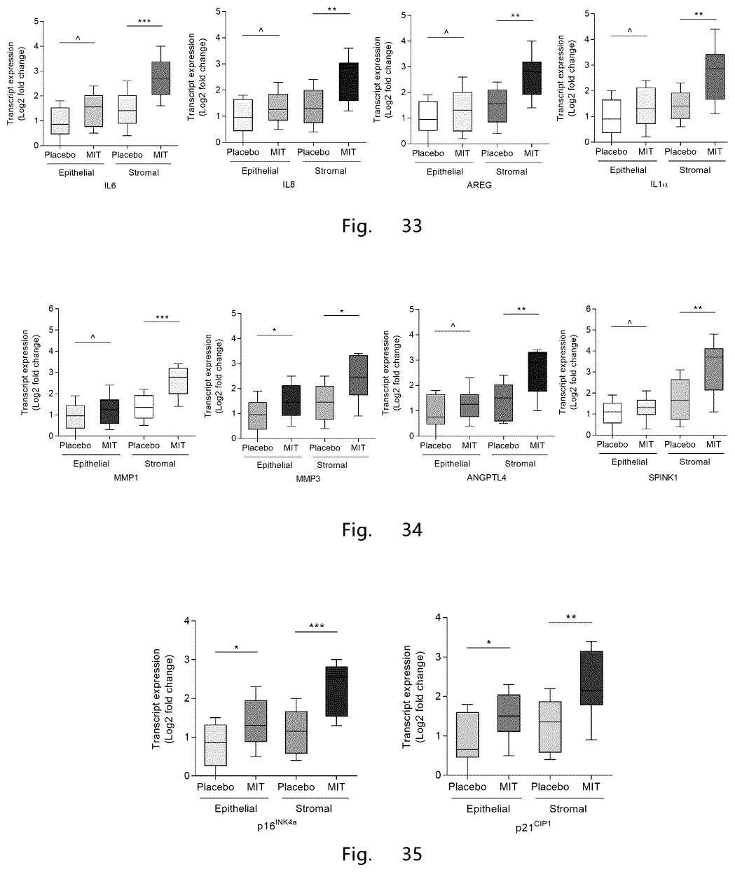

To closely simulate clinical conditions that involve chemotherapy, the inventors designed a preclinical regimen which incorporates genotoxic drugs and/or rutin (FIG. 30, FIG. 31). Two weeks post human cell implantation when stable uptake of tumors by host animals was generally observed, a single dose of MIT or placebo was administered at the 1st day of 3rd, 5th and 7th week until the end of the 8-week regimen (FIG. 31). In contrast to placebo, MIT treatment resulted in remarkably reduced tumor sizes, validating the efficacy of MIT as a cytotoxic agent (FIG. 32). Of note, there was a significant upregulation of typical SASP factors including IL6, IL8, AREG, ILla, MMP1, MMP3, ANGPTL4 and SPINK1, concurrent with the induction of typical senescence markers including p16^{INK4a} p21^{CIP1} and SA-β-GAL, implying development of an in vivo senescence and the SASP in response to MIT treatment (FIG. 33, FIG. 34 and FIG. 35). Interestingly, expression of certain SASP factors such as MMP3, together with the canonical senescence markers including p16^{INK4a} and p21^{CIP1}, was induced by MIT in both stromal and cancer cell populations, suggesting chemotherapy caused comprehensive in vivo senescence, although the SASP profile seemed to be differently developed between these two cell subpopulations (FIG. 34, FIG. 35). SA-β-GAL staining results confirmed considerable tissue senescence induced by MIT, a case in sharp contrast to rutin, which seemed to neither promote nor suppress senescence (FIG. 36), a feature largely consistent with in-vitro observations of the inventors.

The inventors next interrogated whether technically inhibiting development of the full SASP of treatment-damaged stoma could further enhance the therapeutic response of tumors. To this end, rapamycin, one of the well-established senomorphic agents, particularly effective in targeting the development of SASP, was administered since the first dose of preclinical treatment. Simultaneously, rutin was used by the inventors, as a parallel treatment for comparing the intervention effects of both. Though MIT per se caused shrinkage of PC3-only tumors (p<0.001), delivery of senomorphics did not show a remarkable effect (p>0.05) (FIG. 37).

It is noteworthy that these agents did not confer further benefits even when they were combined with MIT (p>0.05), implying the independence of PC3 tumor growth on the tissue-level SASP, specifically in the absence of stromal cells. Strikingly, upon combination of PC3 cells together with their stromal counterparts, the inventors observed markedly increased tumor volumes (p<0.0001), further validating the tumor-promotive effect of stromal cells in vivo (FIG. 37). However, when animals harboring PC3/PSC27 tumors were exposed to MIT, tumor volumes remarkably decreased (35.5%, p<0.001). After coadministration of rapamycin or rutin with MIT as dual treatment, the tumors showed further shrinkage in size, which was extremely significant. Wherein, coadministration of rapamycin and MIT as dual treatments, tumors displayed further shrinkage by 34.1% (p<0.01); coadministration of rutin and MIT as dual treatments, tumors displayed further shrinkage by 48.9% (p<0.0001) (FIG. 37). This result indicates that the combination of rutin with MIT exhibits relatively superior efficacy compared to the combination of rapamycin with MIT.

Notably, when rapamycin and rutin were used together with the chemotherapeutic drug MIT to treat tumors, the intervention achieved the best effect, with tumor volume further reduced compared to MIT monotherapy (68.3%, p<0.0001) (FIG. 37), which was unexpected.

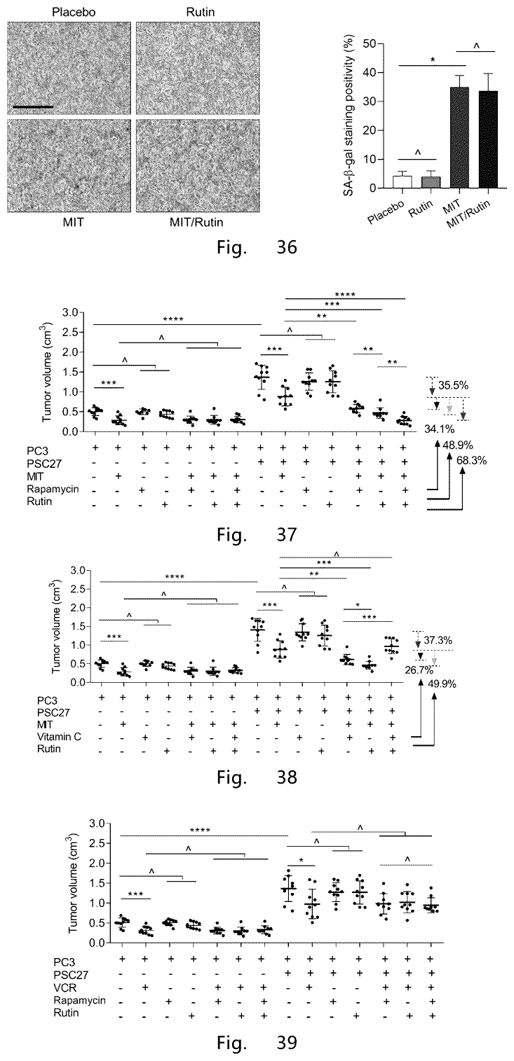

However, when the inventors replaced rapamycin with vitamin C, a natural antioxidant and SASP inhibitor, in combination with rutin and MIT for tumor treatment, they could not replicate these intervention effects, and tumor volume increased, essentially returning to the level observed with MIT monotherapy (FIG. 38). Therefore, even though all are effective SASP inhibitors commonly used in the senescent field, the combined use of rutin with different small molecule compounds and chemotherapeutic drugs can result in markedly different outcomes. The synergistic effect caused by the combined use of rutin and rapamycin provides new options and ideas for future cancer treatment.

Based on this, the inventors investigated whether the therapeutic effects of other chemotherapeutic drugs would also be significantly enhanced under the conditions of combined use with rutin and/or rapamycin. The results indicated that this was not the case; multiple chemotherapeutic drugs did not benefit from the combined use of rutin and/or rapamycin in anti-tumor treatments, for example, vincristine (VCR) did not show such benefits (FIG. 39).

To unmask the mechanism(s) inherently responsible for the SASP-induced cancer resistance, the inventors chose to dissect tumors from animals 7 days after initiation of treatment, a time point prior to development of resistant colonies. In contrast to the placebo, MIT per se caused substantial DNA damage and apoptosis in cancer cells (FIG. 40). Rutin alone caused neither a typical DDR nor enhanced cell death in PC3/PSC27 xenografts, suggesting limited responses of these tumors when animals were exposed to rutin only.

Upon combination with MIT, rutin further increased the percentage of cell apoptosis, implying an augmented cytotoxicity upon coadministration of MIT and rutin. The pattern of in vivo apoptosis was generally consistent with that of tumor regression upon treatment by different agents. Immunohistochemistry (IHC) staining indicated enhanced caspase 3 cleavage, a typical cell apoptosis indicator, when rutin was administered with MIT (FIG. 40). When rutin and rapamycin are used in combination with MIT, DNA damage and apoptosis were further enhanced compared to the combination of rutin and MIT (FIG. 40). This indicates that, under this condition, the clearance of cancer cells in the lesions can achieve optimal results.

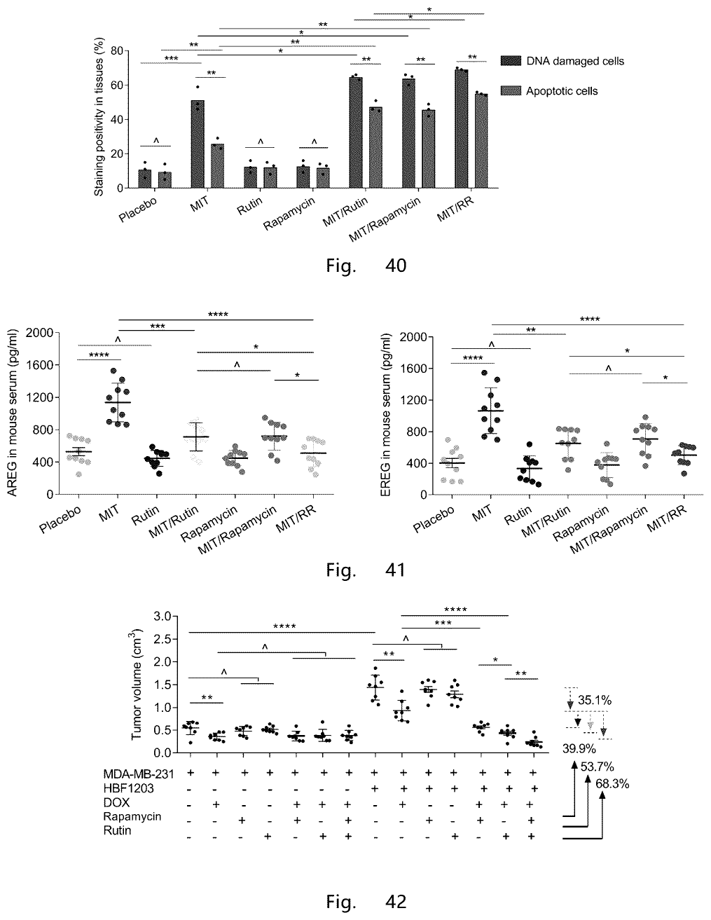

ELISA data suggested that MIT-mediated chemotherapy resulted in elevated levels of circulating AREG and EREG in animals, a pattern that was basically reversed when rutin was delivered (FIG. 41). When rutin is combined with rapamycin, it can further reduce the protein levels of AREG and EREG in the blood of mice, compared to the use of rutin alone with MIT (FIG. 41).

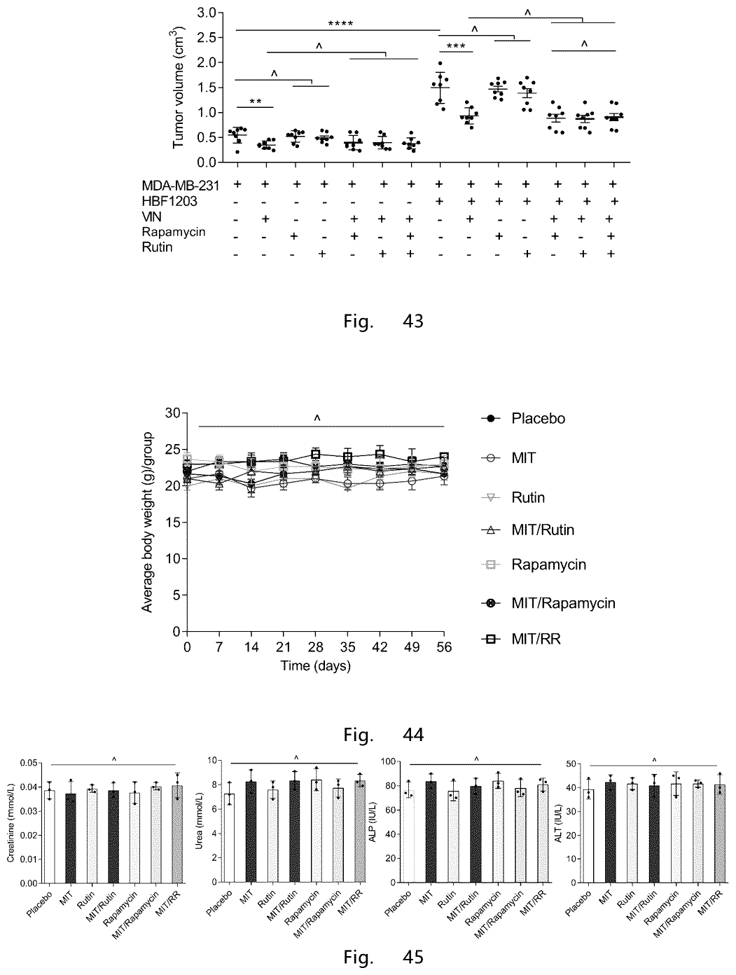

Given the pronounced efficacy of combinational treatment in cancer therapy, the inventors further expanded the study on these drugs and tumor intervention. To confirm breast cancer progressions and treatment results, xenografts comprising MDA-MB-231 (malignant) and HBF1203 (stromal) cells were generated, a combination of human breast-derived cells previously used by the inventors in cancer research. Again, MDA-MB-231/HBF1203 tumors largely reproduced the results of preclinical experiments performed with PCa system under the intervention of specific chemotherapeutic drugs such as doxorubicin (DOX) (FIG. 42). However, not all chemotherapeutic interventions showed this effect; for instance, vincristine (VIN) did not exhibit the characteristics of effective drug combination therapy (FIG. 43).

The findings suggest that the resistance-minimizing effects of the SASP-targeting strategy with a senomorphic agent are not limited to a specific cancer type, but likely applicable to a wide range of solid malignancies, but practically applicable to some certain chemotherapeutic drugs. Overall, the underlying mechanisms determining these differential intervention effects require further in-depth investigation.

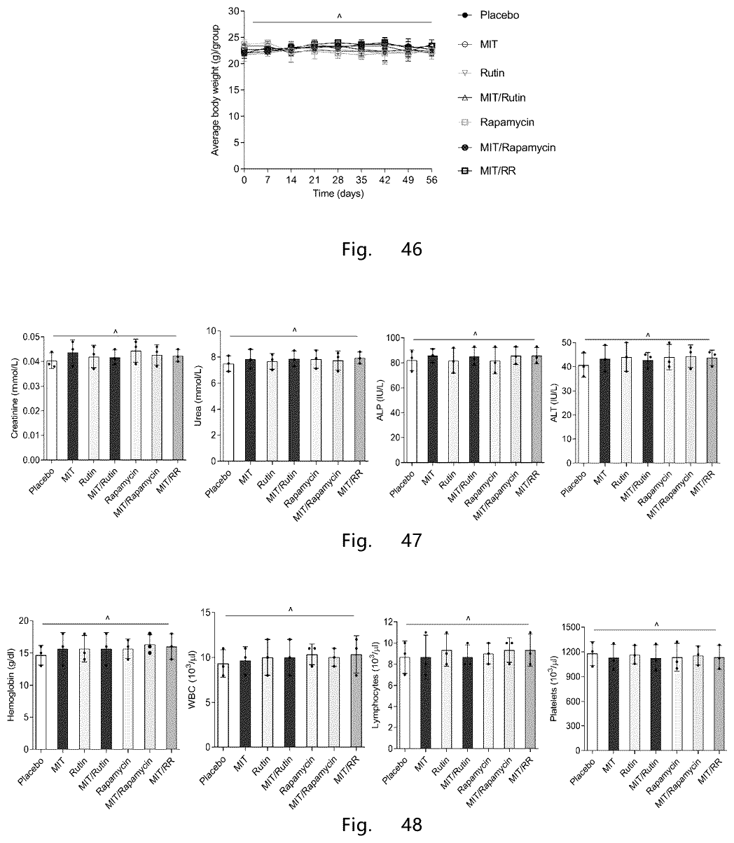

As a crucial step before the translation of modern medicines into clinical practice, the researchers subsequently evaluated the safety of rutin, a novel senomorphics, and rapamycin when used in combination with traditional chemotherapeutic agents for anticancer purposes. The data supported that various physiological results in experimental mice, including daily body weight, liver and kidney toxicity indicators (such as creatinine, urea, ALP, ALT, etc.), and blood components (such as globulin, white blood cells, lymphocytes, platelets, etc.), did not exhibit significant perturbations (FIG. 44-48).

These findings suggest that rutin is a highly effective and safe natural small-molecule senomorphics. Rapamycin, in combination with rutin, synergistically enhances the efficacy of chemotherapeutic drugs, demonstrating promising prospects for future clinical applications in cancer treatment.

### Example 6. Analysis of Antitumor Effects on Different Drug Combinations

In preliminary extensive screening studies conducted by the inventors, the effects of various drug combinations on anticancer effects were compared. Some drug combinations did not show significantly improved effects when used together, while others led to a decrease in anticancer effects.

The combinations of drugs resulting in decreased anticancer effects and the corresponding anticancer results are listed in Table 1. The administration methods in the experiments were consistent with the aforementioned preclinical treatment trials, and components not specified in the table were administered according to the doses provided in the aforementioned preclinical treatment trials.

TABLE 1

| | Effects on xenografts in mice |
|---|---|
| | transplanted with prostate cancer |
| Combinations | PC3 cells |
| Rutin + Apigenin (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Mitoxantrone | reduces the anti-tumor effect. |
| Rutin + 5Z-7-oxozeaenol (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Mitoxantrone | reduces the anti-tumor effect. |
| Rutin + SB203580 (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Mitoxantrone | reduces the anti-tumor effect. |
| Rutin + Metformin (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Mitoxantrone | reduces the anti-tumor effect. |
| Rutin + Baicalein (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Mitoxantrone | reduces the anti-tumor effect. |
| Rutin + Kaempferol (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Mitoxantrone | reduces the anti-tumor effect. |
| Apigenin (500 μl, administered in a | It has no positive effect on |
| dose of 10 mg/kg for 3 times with a | enhancing the anti-tumor effect of |
| total of 30 mg/kg body weight) + | rutin + mitoxantrone, but instead |
| Rapamycin + Mitoxantrone | reduces the anti-tumor effect. |
| 5Z-7-oxozeaenol (500 μl, | It has no positive effect on |
| administered in a dose of 10 mg/kg | enhancing the anti-tumor effect of |
| for 3 times with a total of 30 mg/kg | rutin + mitoxantrone, but instead |
| body weight) + Rapamycin + | reduces the anti-tumor effect. |
| Mitoxantrone | |
| SB203580 (500 μl, administered in a | It has no positive effect on |
| dose of 10 mg/kg for 3 times with a | enhancing the anti-tumor effect of |
| total of 30 mg/kg body weight) + | rutin + mitoxantrone, but instead |
| Rapamycin + Mitoxantrone | reduces the anti-tumor effect. |
| Metformin (500 μl, administered in a | It has no positive effect on |
| dose of 10 mg/kg for 3 times with a | enhancing the anti-tumor effect of |
| total of 30 mg/kg body weight) + | rutin + mitoxantrone, but instead |
| Rapamycin + Mitoxantrone | reduces the anti-tumor effect. |
| Baicalein (500 μl, administered in a | It has no positive effect on |
| dose of 10 mg/kg for 3 times with a | enhancing the anti-tumor effect of |
| total of 30 mg/kg bodyweight) + | rutin + mitoxantrone, but instead |
| Rapamycin + Mitoxantrone | reduces the anti-tumor effect. |
| Kaempferol (500 μl, administered in | It has no positive effect on |
| a dose of 10 mg/kg for 3 times with a | enhancing the anti-tumor effect of |
| total of 30 mg/kg body weight) + | rutin + mitoxantrone, but instead |
| Rapamycin + Mitoxantrone | reduces the anti-tumor effect. |

Therefore, most drug combinations do not achieve pharmaceutically significant improvements. After extensive screening and combination studies, the present disclosure has obtained a combination of rutin, rapamycin and mitoxantrone with synergistic effects.

Above-described examples only show several embodiments of the present disclosure, which are described specifically and in detail. However, it should not be understood as a limiting patent scope of the present disclosure. It should be noted that those skilled in the art can make some adjustments and improvements without departing from the concept of the present disclosure and all these forms are within the scope of protection of the present disclosure. Therefore, the scope of patent protection in the present disclosure should be determined by the appended claims. Simultaneously, each reference provided herein is incorporated by reference to the same extent as if each reference was individually incorporated by reference.

1. A method for specifically-targeted inhibiting senescence-associated secretory phenotype, inhibiting tumors and/or reversing cancer resistance, comprising administering rutin and rapamycin or derivatives thereof, combined with a chemotherapeutic drug; wherein the chemotherapeutic drug is capable of inducing senescence-associated secretory phenotype after administration, comprising mitoxantrone or bleomycin; the derivatives comprise pharmaceutically acceptable salts, esters, isomers, solvates or prodrugs of rutin and rapamycin.

2. The method according to claim 1, wherein, the senescence-associated secretory phenotype is a senescence-associated secretory phenotype caused by DNA damage; the DNA damage is a DNA damage induced by a chemotherapeutic drug.

3. The method according to claim 1, wherein, in the composition, the rutin or rapamycin or derivatives thereof is also used for:
inhibiting expression of the full spectrum senescence-associated secretory phenotype (SASP); inhibiting expression of the full spectrum SASP without affecting cellular senescence;
interfering with the interaction of ATM with HIF1α and TRAF6, inhibiting acute stress-associated phenotype;
eliminating malignancy of cancer cells conferred by senescent stromal cells through paracrine pathways;
increasing apoptosis rate of cancer cells; and/or
inhibiting components of the senescence-associated secretory phenotype, comprising IL8, IL6, ILla, IL1b, CXCL3, MMP3 and GM-CSF.

4. The method according to claim 1, wherein, the tumor comprises: prostate cancer, breast cancer, lung cancer, colorectal cancer, gastric cancer, liver cancer, pancreatic cancer, bladder cancer, skin cancer, kidney cancer, esophageal cancer, bile duct cancer and brain cancer.

5. The method according to claim 1, wherein, the chemotherapeutic drug is mitoxantrone, wherein the weight ratio of mitoxantrone to rutin to rapamycin is 1:20˜80:20˜80.

6. A pharmaceutical composition or drug kit for specifically-targeted inhibiting senescence-associated secretory phenotype, inhibiting tumors and/or reversing cancer resistance, comprising: rutin and rapamycin or derivatives thereof, and a chemotherapeutic drug; wherein the chemotherapeutic drug is capable of inducing senescence-associated secretory phenotype after administration, comprising mitoxantrone or bleomycin; the derivatives comprise pharmaceutically acceptable salts, esters, isomers, solvates or prodrugs of rutin and rapamycin.

7. A method of preparing a pharmaceutical composition or drug kit for inhibiting tumors and/or reversing cancer resistance; comprising: mixing rutin and rapamycin or derivatives thereof and a chemotherapeutic drug; or placing rutin and rapamycin or derivatives thereof and a chemotherapeutic drug in the same drug kit; wherein, the chemotherapeutic drug is capable of inducing senescence-associated secretory phenotype after administration, comprising mitoxantrone or bleomycin; the derivatives comprise pharmaceutically acceptable salts, esters, isomers, solvates or prodrugs of rutin and rapamycin.

8. A method for specifically-targeted inhibiting senescence-associated secretory phenotype, comprising administering rutin and rapamycin or derivatives thereof, combined with a chemotherapeutic drug; the rutin and rapamycin or derivatives thereof interfere with the interaction of ATM with HIF1α and TRAF6, inhibit acute stress-associated phenotype; eliminate malignancy of cancer cells conferred by senescent stromal cells through paracrine pathways; and/or, increase apoptosis rate of cancer cells; the derivatives comprise pharmaceutically acceptable salts, esters, isomers, solvates or prodrugs of rutin and rapamycin.

9. A method of screening a potential substance for promoting rutin and rapamycin to target and inhibit senescence-associated secretory phenotype, inhibit tumors and/or reverse cancer resistance, wherein the method comprises:
(1) providing a system of tumor microenvironment, wherein the system comprises tumor cells;
(2) treating the system of (1) with a chemotherapeutic drug, inducing a senescence-associated secretory phenotype in the tumor microenvironment; wherein the chemotherapeutic drug is capable of inducing senescence-related secretory phenotype after administration, comprising mitoxantrone or bleomycin; administering rutin and rapamycin before, during or after inducing the senescence-related secretory phenotype in the tumor microenvironment; and
(3) adding the candidate substance to the system in (2) and observing its effect on the tumor microenvironment system; if the candidate substance statistically promotes rutin and rapamycin to inhibit the senescence-associated secretory phenotype, inhibit tumors and/or reverse cancer resistance, then the candidate substance is a potential substance that can be used in combination with rutin and rapamycin to inhibit tumors.

10. A method for screening a potential substance for inhibiting senescence-associated secretory phenotype, wherein the method comprises:
(1) providing a system of stromal cells, inducing the senescence-associated secretory phenotype in the system; administering rutin and rapamycin before, during or after inducing the senescence-related secretory phenotype in the system;
(2) adding the candidate substance to the system of (1) and observing its effect on the system of stromal cells. If the candidate substance statistically promotes rutin and rapamycin to inhibit the senescence-associated secretory phenotype, then the candidate substance is a potential substance that can be used in combination with rutin and rapamycin to inhibit the senescence-associated secretory phenotype.

11. The method according to claim 5, wherein, the weight ratio of mitoxantrone to rutin to rapamycin is 1:30˜70:30˜70.

12. The method according to claim 11, wherein, the weight ratio of mitoxantrone to rutin to rapamycin is 1:40˜60:40˜60.

13. The pharmaceutical composition or drug kit according to claim 6, wherein, when mitoxantrone, rutin and rapamycin are used in combination, the weight ratio of mitoxantrone to rutin to rapamycin is 1:20˜80:20˜80.

14. The pharmaceutical composition or drug kit according to claim 13, wherein, the weight ratio of mitoxantrone to rutin to rapamycin is 1:30˜70:30˜70.

15. The pharmaceutical composition or drug kit according to claim 14, wherein, the weight ratio of mitoxantrone to rutin to rapamycin is 1:40˜60:40˜60.

16. The method according to claim 7, wherein, when mitoxantrone, rutin and rapamycin are mixed or used in combination, the weight ratio of mitoxantrone to rutin to rapamycin is 1:20˜80:20˜80.
